(19) United States
(12) Patent Application Publication
(10) Pub. No.: US 2011/0172490 A1
(43) Pub. Date: Jul. 14, 2011

(54) INSERTION PORTION HOLDER, ENDOSCOPE APPARATUS, AND METHOD OF BUNDLING INSERTION PORTION

(75) Inventor: Yoshihisa ISHIKAWA, Tokyo (JP)

(73) Assignee: OLYMPUS CORPORATION, Tokyo (JP)

(21) Appl. No.: 12/948,131

(22) Filed: Nov. 17, 2010

(30) Foreign Application Priority Data
Jan. 8, 2010 (JP) 2010-003196

(51) Int. Cl. A61B 1/00; B23P 11/00

(52) U.S. Cl. 600/102; 29/428

(57) ABSTRACT

An insertion portion holder includes a plane portion formed in a flat shape with an elastic member, the plane portion including: a first through hole formed on the plane portion, for passing therethrough an insertion portion having flexibility extended from an endoscope operation portion; and a second through hole formed on the plane portion adjacently to the first through hole, for holding a distal end side part of the insertion portion.

### CROSS REFERENCE TO RELATED APPLICATION

This application claims the benefit of Japanese Application No. 2010-003196 filed in Japan on Jan. 8, 2010, the contents of which are incorporated herein by this reference.

### BACKGROUND OF THE INVENTION

1. Field of the Invention

The present invention relates to an insertion portion holder, an endoscope apparatus, and a method of bundling an insertion portion.

2. Description of Related Art

In recent years, endoscopes having an elongated insertion portion have been used in medical fields and industrial fields. The endoscope apparatuses used in industrial fields are required to have portability. Japanese Patent Application Laid-Open Publication No. 2004-321243 discloses an endoscope apparatus which is mainly composed of an endoscope containing an image pickup device at a distal end portion of an elongated insertion portion, and an apparatus main body having a monitor.

The endoscope in the endoscope apparatus includes, at a proximal end portion of the insertion portion, an operation portion including a lever for bending a bending portion of the insertion portion. The apparatus main body includes a power source portion which supplies power source/electric power to the endoscope, a light source portion which supplies illumination light for illuminating a region to be observed, an electric circuit which drives the image pickup device, and an image processing portion which generates a video signal from an image signal outputted from an image pickup device.

In order to improve the portability, the endoscope apparatus is provided with an attaching/detaching portion which allows the operation portion of the endoscope to be detachably attached to and to be detached from one side portion of a housing configuring the apparatus main body. In addition, the endoscope apparatus is provided with a belt, not shown, and the housing of the apparatus main body includes a housing-side clamp to which a belt clamp of the belt can be detachably attached.

According to the endoscope apparatus thus configured, an operator wears the apparatus main body with the operation portion of the endoscope attached to the apparatus main body, which enables the operator to easily carry the endoscope apparatus by slinging the apparatus over his or her shoulder.

However, when the operator carries the endoscope apparatus by slinging the apparatus over his or her shoulder, if the insertion portion extended from the operation portion is hanging low, the operator may accidentally step on the insertion portion or may bump the insertion portion on a wall, an obstacle, or the like, during carrying.

If the distal end side part of the insertion portion is stepped on or bumped, the image pickup device, an optical lens, and the like provided at the distal end portion are damaged, which may cause a defect in an observation image, or bending pieces configuring the bending portion is deformed, which may disturb bending operation.

United States Patent Application Publication No. 2008/0208002 discloses an endoscope which prevents such a problem that an optical lens, an image pickup device, or light emitting elements incorporated in a distal end portion may be damaged when an object is placed on an insertion portion, in particular, on the distal end portion of the insertion portion, or an operator and the like steps on the insertion portion. The endoscope includes, at a front face portion of an operation portion main body, a holding member including a holding portion for holding the insertion portion. The holding member includes, at a center of an upper end side thereof, a holding hole which serves as the holding portion. The inner diameter of the holding hole is so formed as to be larger than the outer diameter of the insertion portion by a predetermined dimension. The distal end portion of the insertion portion, a bending portion, or a distal end side of a flexible tube portion is arranged in the holding hole, and thereby the insertion portion is held by the holding member. As a result, the distal end side part of the insertion portion is arranged in a space. Therefore, it is possible to prevent the distal end portion of the insertion portion and the like from bumping on a wall or an obstacle or to prevent a distal end side part of the insertion portion to be placed under an object or to be stepped on, during carrying of the endoscope apparatus. However, when the operator carries the endoscope apparatus by slinging the apparatus over his or her shoulder, the insertion portion extended from the operation portion hangs low.

In addition, United States Patent Application Publication No. 2009/0106948 discloses a tie including a strap having one end side portion and the other end side portion, wherein a hole is formed on the one end side portion. According to the tie, after an operator passed an elongated flexible tubular body through the hole and arranged the tubular body at a predetermined position, the operator winds the tubular body in a coil shape. Thereafter, the operator wraps the strap of the tie around a bundle of the tubular body wound in a coil shape, and fixes the other end side portion of the strap to the one end side portion of the strap, thereby holding the elongated flexible tubular body in the coil shape. In addition, as shown in FIG. 17, according to the tie, after winding an elongated flexible probe which is extended from a grasping operation portion of an examination instrument, an operator can bundle the probe with the tie. However, in the tied state, the probe including a coil-shaped portion swings with respect to the grasping operation portion.

### SUMMARY OF THE INVENTION

An insertion portion holder according to the present invention includes a plane portion formed in a flat shape with an elastic member, the plane portion including a first through hole formed on the plane portion, for passing therethrough an insertion portion having flexibility extended from an endoscope operation portion; and a second through hole formed on the plane portion adjacently to the first through hole, for holding a distal end side part of the insertion portion.

The above and other objects, features and advantages of the invention will become more clearly understood from the following description referring to the accompanying drawings.

### BRIEF DESCRIPTION OF THE DRAWINGS

FIGS. 1 to 9 relate to an endoscope apparatus including an insertion portion holder.

FIG. 1 is a view illustrating a configuration of the endoscope apparatus.

FIG. 2A is a front view of the insertion portion holder.

FIG. 2B is a view illustrating a second through hole to be deformed by pressing a pressing portion included in the insertion portion holder by the finger.

FIG. 3 is a view illustrating an endoscope main body including an operation-portion-side attaching/detaching portion which is included in an attaching/detaching mechanism.

FIG. 4 is a view illustrating an apparatus main body including a main-body-side attaching/detaching portion which is included in the attaching/detaching mechanism.

FIG. 5 is view illustrating a held state where a distal end side part of the insertion portion is held by a holding portion of the insertion portion holder retained at a base portion of a break prevention member, and a relationship between an operation portion and the insertion portion when the operation portion is lifted in the held state.

FIG. 6 illustrates a held state where, after the insertion portion is rolled up into a spiral to form a spiral-shaped insertion portion, the distal end side part of the insertion portion is held by the holding portion of the insertion portion holder retained at the base portion, and a relationship between the operation portion and the insertion portion at the time that a part of the spiral-shaped insertion portion and grasping portion are grasped and the operation portion is lifted.

FIG. 7 is a view illustrating a state where the insertion portion is held by the holding portion of the insertion portion holder such that the distal end side part of the insertion portion is directed in a direction of the grasping portion of the operation portion.

FIG. 8 is a view illustrating a procedure of causing the holding portion of the insertion portion holder arranged at a halfway position of the insertion portion to hold the distal end side part of the insertion portion and thereafter making transition to the held state shown in FIG. 5.

FIG. 9 is a view illustrating a procedure of forming the spiral-shaped insertion portion from the held state shown in FIG. 6.

FIGS. 10 to 14 relate to a winding portion holding portion configured by attaching an endoscope main body and an apparatus main body integrally to each other.

FIG. 10 is a view illustrating a procedure of configuring the endoscope apparatus in which the endoscope main body and the apparatus main body are integrally attached to each other.

FIG. 11 is a perspective view illustrating the endoscope apparatus in which the endoscope main body and the apparatus main body are integrally attached to each other.

FIG. 12 is a side view illustrating a procedure of hanging the formed spiral-shaped insertion portion on the operation portion of the endoscope main body integrally attached to the apparatus main body.

FIG. 13 is a perspective view illustrating a procedure of hanging the formed spiral-shaped insertion portion on the endoscope apparatus.

FIG. 14 is a view illustrating a relationship between the operation portion and the insertion portion in a state where the formed spiral-shaped insertion portion is hung on the endoscope apparatus.

FIGS. 15 and 16 relate to another procedure of hanging the spiral-shaped insertion portion on the endoscope apparatus.

FIG. 15 is a view illustrating procedures of forming the spiral-shaped insertion portion with the insertion portion holder arranged at a halfway position of the insertion portion and of moving the insertion portion holder to the operation portion side.

FIG. 16 is a view illustrating a procedure of causing the holding portion of the insertion portion holder moved to the operation portion side to hold the distal end side part of the insertion portion.

FIGS. 17 and 18 relate to another procedure of hanging the spiral-shaped insertion portion on the endoscope apparatus.

FIG. 17 is a perspective view illustrating a procedure of rotating the insertion portion holder arranged at a halfway position of the insertion portion around an axis of the insertion portion and causing the holding portion of the insertion portion holder to hold the distal end side part of the insertion portion.

FIG. 18 is a side view illustrating the procedure of rotating the insertion portion holder arranged at the halfway position of the insertion portion around the axis of the insertion portion and causing the holding portion of the insertion portion holder to hold the distal end side part of the insertion portion.

FIG. 19 relates to yet another procedure of hanging the spiral-shaped insertion portion on the endoscope apparatus, and is a view illustrating a procedure of passing the insertion portion through a first through hole of the insertion portion holder arranged at the halfway position of the insertion portion several times and forming the spiral-shaped insertion portion.

FIG. 20 is a view illustrating another exemplary configuration of the insertion portion holder.

FIG. 21 is view illustrating a procedure of hanging the spiral-shaped insertion portion on the endoscope apparatus using the insertion portion holder having the configuration shown in FIG. 20.

FIG. 22 is a view illustrating another exemplary configuration of the insertion portion holder.

FIG. 23 is a view illustrating yet another exemplary configuration of the insertion portion holder.

FIG. 24 is a view illustrating a procedure of hanging the spiral-shaped insertion portion on the endoscope apparatus using the insertion portion holder having the configuration shown in FIG. 23.

FIGS. 25 and 26 relate to an endoscope having a tying member for bundling the spiral-shaped insertion portion.

FIG. 25 is a view illustrating a configuration and working of the tying member.

FIG. 26 is a view illustrating the spiral-shaped insertion portion bundled using the tying member.

FIG. 27 is a view illustrating the endoscope apparatus configured by electrically connecting a display device to the operation portion and the working of the tying member provided to the endoscope apparatus.

### DETAILED DESCRIPTION OF PREFERRED EMBODIMENTS

Hereinafter, embodiments of the present invention will be described with reference to the drawings.

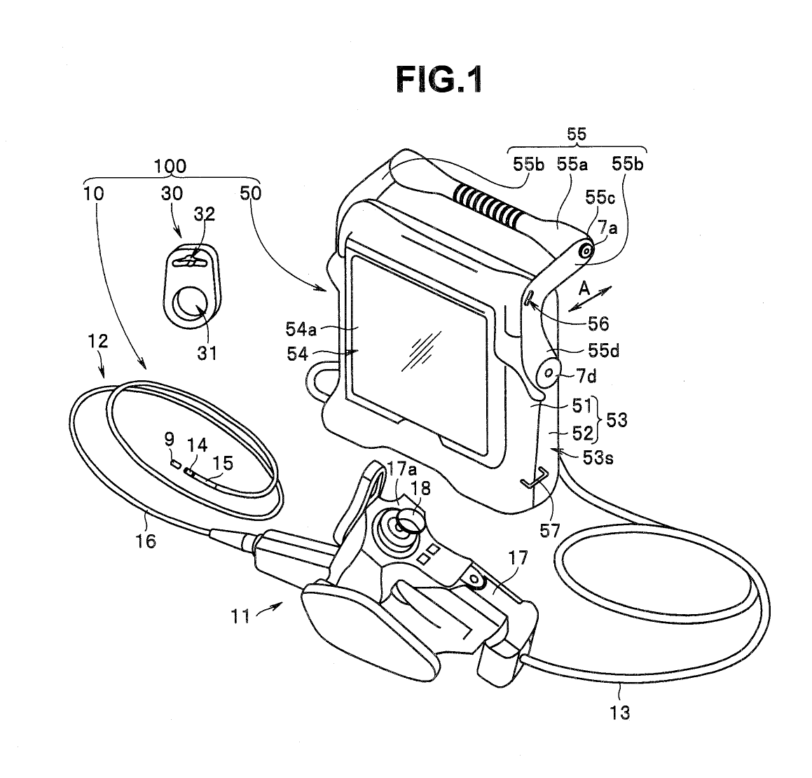

One embodiment of the present invention will be described with reference to FIGS. 1 to 9.

As shown in FIG. 1, an endoscope apparatus 100 is configured by mainly including an endoscope main body 10, an insertion portion holder 30, and an apparatus main body 50.

The endoscope main body 10 includes an endoscope operation portion (hereinafter shortly referred to as operation portion) 11 which is a first housing portion. The apparatus main body 50 includes an exterior portion 53 which is a second housing portion. The reference numeral 9 denotes an endoscope adapter. The adapter 9 is configured to be detachably attached to a distal end side of an endoscope insertion portion (hereinafter shortly referred to as insertion portion) 12 which is provided so as to be extended from the operation portion 11.

In the present embodiment, the endoscope main body 10 includes the operation portion 11, the insertion portion 12 and a universal cord 13 which are extended from the operation portion 11. The insertion portion 12 is elongate and includes a distal end portion 14, a bending portion 15, and a flexible tube portion 16 in a linked manner in this order from the distal end. The distal end portion 14 incorporates an image pickup unit (not shown) including an image pickup device such as a CCD, not shown, as an observation portion for picking up an image of a region to be examined which is observed through the endoscope adapter 9. The bending portion 15 includes a plurality of bending pieces in a linked manner, for example, and is configured to be bent in up, down, left, and right directions. The flexible tube portion 16 has a long length and a preset elasticity. Therefore, the flexible tube portion 16 can be rolled up into a spiral having a preset inner diameter.

An image pickup system signal line, a bending wire, a power supply line, and the like are passed through the insertion portion 12. The image pickup system signal line is connected to the image pickup unit. The bending wire causes the bending portion 15 to perform bending operation. The endoscope adapter 9 includes LEDs for illuminating the region to be examined The power supply line supplies power to the LEDs, for example.

As described above, the endoscope main body 10 has a configuration in which the endoscope adapter 9 is detachably attached to the distal end portion 14 of the insertion portion 12. However, the endoscope main body 10 may be configured to include an illumination portion and an observation portion in the distal end portion 14. In addition, the endoscope adapter 9 may include a light guide fiber. In such a configuration, the light guide fiber for transmitting illumination light is passed through the insertion portion 12, instead of the power supply line. Furthermore, as described above, the insertion portion 12 includes the bending portion 15. However, the configuration of the insertion portion 12 is not limited to one including the bending portion 15. The insertion portion 12 may have a configuration in which the flexible tube portion 16 is provided in a manner linked with the distal end portion 14.

The operation portion 11 includes a grasping portion 17 to be grasped by an operator. The operation portion 11 includes a bending operation lever 18. The bending operation lever 18 is arranged at a position where the operator can perform bending operation of the bending portion 15 while grasping the grasping portion 17. The bending operation lever 18 is provided upright on a top surface of the grasping portion 17. The bending operation lever 18 is tiltable in at least four directions. When the operator changes the tilting direction of the lever, the bending portion 15 is bent in one direction among up-down/left-right four directions.

In addition to the image pickup system signal line and the power supply line which supplies power to the LEDs, a power supply line for motor which supplies power to a driving motor provided in the operation portion 11 is passed through the universal cord 13. In a case where the endoscope does not include a bending portion, there is no need to provide the bending operation lever 18, the driving motor, and the power supply line for motor.

A proximal end of the universal cord 13 extended from the operation portion 11 is connected to the exterior portion 53 configuring the apparatus main body 50.

The exterior portion 53 is configured by attaching a first exterior forming member (hereinafter shortly referred to as first exterior portion) 51 and a second exterior forming member (hereinafter shortly referred to as second exterior portion) 52 integrally to each other. The exterior portion 53 of the present embodiment is formed in a flat rectangular parallelepiped shape, the thickness dimension of which is reduced. A monitor 54 as a display device is provided on one surface of the first exterior portion 51. An endoscopic image picked up by the image pickup unit of the endoscope main body 10 is displayed on a screen 54a of the monitor 54.

In the present embodiment, the apparatus main body 50 configures what is called a flat-screen monitor. In the exterior portion 53 having such a configuration, the one surface on which the monitor 54 is arranged is defined as a front surface, and the rear surface side of the one surface is defined as a back surface.

Inside the exterior portion 53, electric parts such as a CPU, a battery unit as a power source portion, and the like are provided. The CPU controls a circuit which generates a signal for driving the image pickup device, a circuit which generates a video signal based on an electric signal transmitted from the image pickup unit, for example. The battery unit supplies power to the LEDs, the monitor 54, or the driving motor.

A handle 55 serving also as a stand is attached to the exterior portion 53. The handle 55 improves portability of the apparatus main body 50, and allows the apparatus main body 50 to be used in a standing position.

The handle 55 includes a handle main body 55a and a pair of arm portions 55b formed in a substantially L-shape. Each of one end portions 55c of the arm portions 55b is integrally fixed on each end surface of the handle main body 55a with a fixing bolt 7a. On the other hand, each of the other end portions 55d of the arm portions 55b is attached on each of both side surfaces 53s adjacent to the screen 54a with a clamping member 7b.

The reference numeral 56 denotes a belt attaching hole. The belt attaching hole 56 is formed at each flexed portion of the arm portions 55b. A hook of a belt is attached to each of the belt attaching holes 56. In addition, a belt attaching clevis 57 is provided at each of the lower portions of the both side surfaces 53s of the exterior portion 53. The hook of the belt to be described later is attached to each of the belt attaching clevises 57.

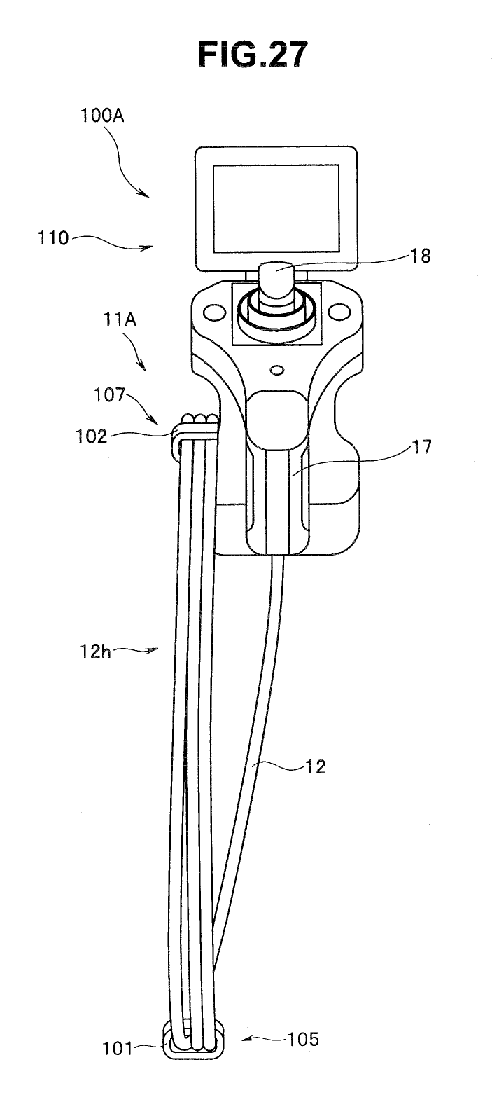

In the present embodiment, the apparatus main body 50 includes the monitor 54 as a display device. However, as shown in FIG. 27, for example, an endoscope apparatus 100A may be configured by electrically connecting a single display device 110 as a second housing to an operation portion 11A. Since the endoscope apparatus 100A includes the display device 110 at the operation portion 11A, the CPU and the like may be provided in the operation portion 11A. Other configurations are the same as those in the above described embodiment. The same components are attached with the same reference numerals and descriptions thereof will be omitted.

The endoscope apparatus 100 of the present embodiment is configured in consideration of the portability. Therefore, a belt is attached to the apparatus main body 50 and an attaching/detaching mechanism 60 for attaching the endoscope main body 10 and the apparatus main body 50 integrally to each other, and the insertion portion holder 30 for holding the insertion portion 12 in place and preventing the insertion portion from hanging low.

Here, the insertion portion holder 30 and the attaching/detaching mechanism 60 are described.

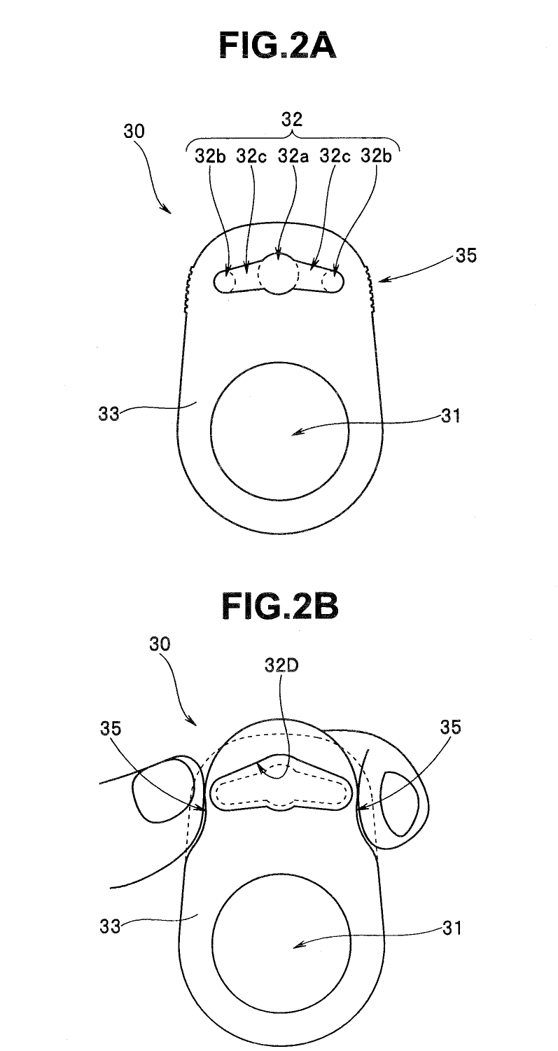

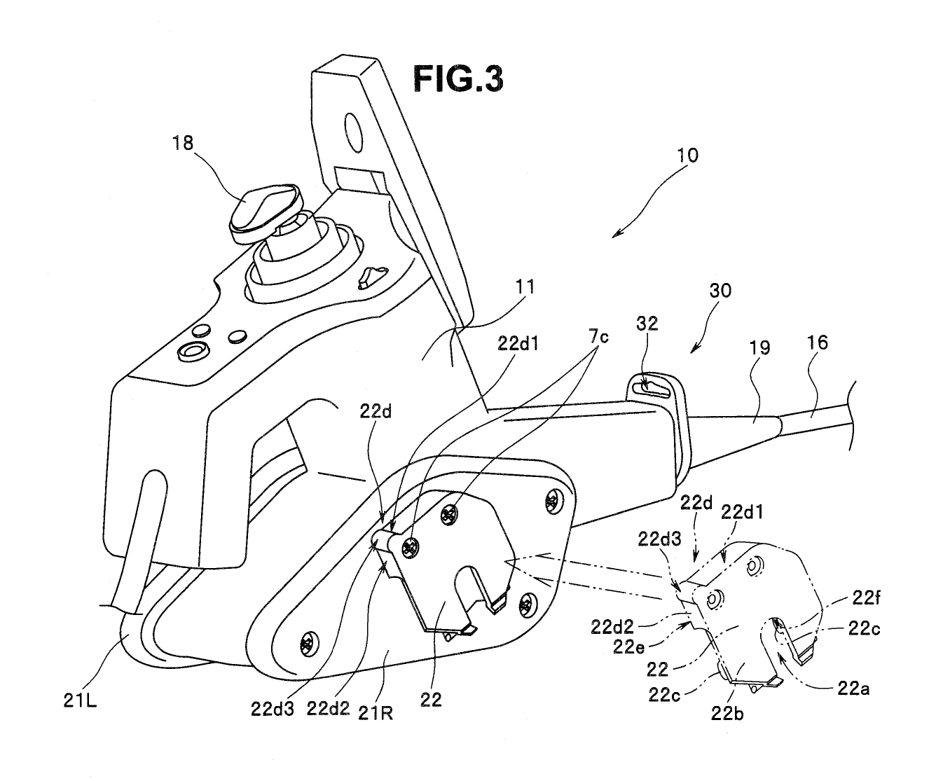

First, description is made on the insertion portion holder 30. The insertion portion holder 30 is made of elastic silicone rubber, for example, and is formed in a flat and substantially elliptical shape. As shown in FIGS. 1 and 2A, the insertion portion holder 30 includes a first through hole 31 and a second through hole 32 on a plane portion 33.

The first through hole 31 has a substantially circular shape. The inner diameter of the first through hole 31 is so formed as to be larger than an outer diameter of the insertion portion 12 and the first through hole is configured to be detachably retained by an elastic force at a base portion 19a, for example, of a break prevention member 19 configuring the operation portion 11.

Therefore, the insertion portion holder 30 is movable in a longitudinal axis direction of the insertion portion 12 and rotatable around the longitudinal axis with the insertion portion 12 passed through the first through hole 31 of the insertion portion holder 30. By moving the insertion portion holder 30 in the longitudinal direction toward the operation portion 11, the insertion portion holder can be fixedly arranged at the base portion 19a of the break prevention member 19 so as to be integral with the base portion 19a.

The second through hole 32 has a long hole shape. The shape of the second through hole is different from that of the first through hole 31. The second through hole 32 is configured to be able to hold two kinds of insertion portions 12 having different outer diameter dimensions, for example. That is, the second through hole 32 can hold the distal end side of the distal end portion 14 and the distal end side of the flexible tube portion 16 of each of the two kinds of insertion portions 12. In the present embodiment, the second through hole 32 holds the distal end side of the flexible tube portion 16.

The second through hole 32 includes a first insertion portion holding portion (hereinafter shortly referred to as first holding portion) 32a, a second insertion portion holding portion (hereinafter shortly referred to as second holding portion) 32b, and a communicating hole 32c. The first holding portion 32a holds the insertion portion having a first diameter. The second holding portion 32b holds the insertion portion having a second diameter smaller than the first diameter. The communicating hole 32c allows communication between the second holding portion 32b and the first holding portion 32a.

The first holding portion 32a has a substantially circular shape. The first holding portion 32a is formed at the center of the second through hole 32 which is a long hole. The inner diameter of the first holding portion 32a is smaller than the first diameter by a preset dimension. As a result, when the distal end side of the flexible tube portion 16 as the insertion portion having the first diameter is arranged in the first holding portion 32a, for example, the insertion portion is held integrally in the first holding portion 32a by an elastic force of the insertion portion holder 30.

The second holding portion 32b has a substantially semicircular shape. The second holding portion 32b is formed on each of the both side portions of the second through hole 32 so as to sandwich the first holding portion 32a. The inner diameter of the second holding portion 32b is smaller than the second diameter by a preset dimension. As a result, when the distal end side of the flexible tube portion 16 as the insertion portion having the second diameter is arranged in the second holding portion 32b, for example, the insertion portion is held integrally in the second holding portion 32b by the elastic force of the insertion portion holder 30.

The communicating hole 32c is so formed as to be gradually wider from the end side to the center. The insertion portion holder 30 has a pressing portion 35 on an outer peripheral surface. As shown in FIG. 2B, when an operator presses the pressing portion 35 with his or her fingers, for example, the insertion portion holder 30 is deformed as shown with the dashed lines. That is, when the operator presses the pressing portion 35, the diameters of the holding portions 32a, 32b are expanded and the width of the communicating hole 32c is extended, which provides a second through hole 32D.

Accordingly, when arranging the insertion portion having the first diameter in the first holding portion 32a, or arranging the insertion portion having the second diameter in the second holding portion 32b, the operator obtains the second through hole 32D by pressing the pressing portion 35, thereby easily arranging the insertion portion at the desired holding portion 32a or 32b.

The pressing portion 35 includes a plurality of concavo-convex portions. The concavo-convex portions serve both as an anti-slip portion for preventing the fingers from slipping and a notification portion for notifying the position of the pressing portion 35.

Furthermore, the first through hole 31 is formed in a substantially circular shape in the present embodiment. However, the shape of the first through hole 31 is not limited to the circular shape. The shape of the first through hole 31 may be formed in a shape in accordance with the shape of the base portion 19a of the break prevention member 19, or may be formed in a polygonal shape, an elliptical shape or the like such that the first through hole can hold the insertion portion by the elastic force.

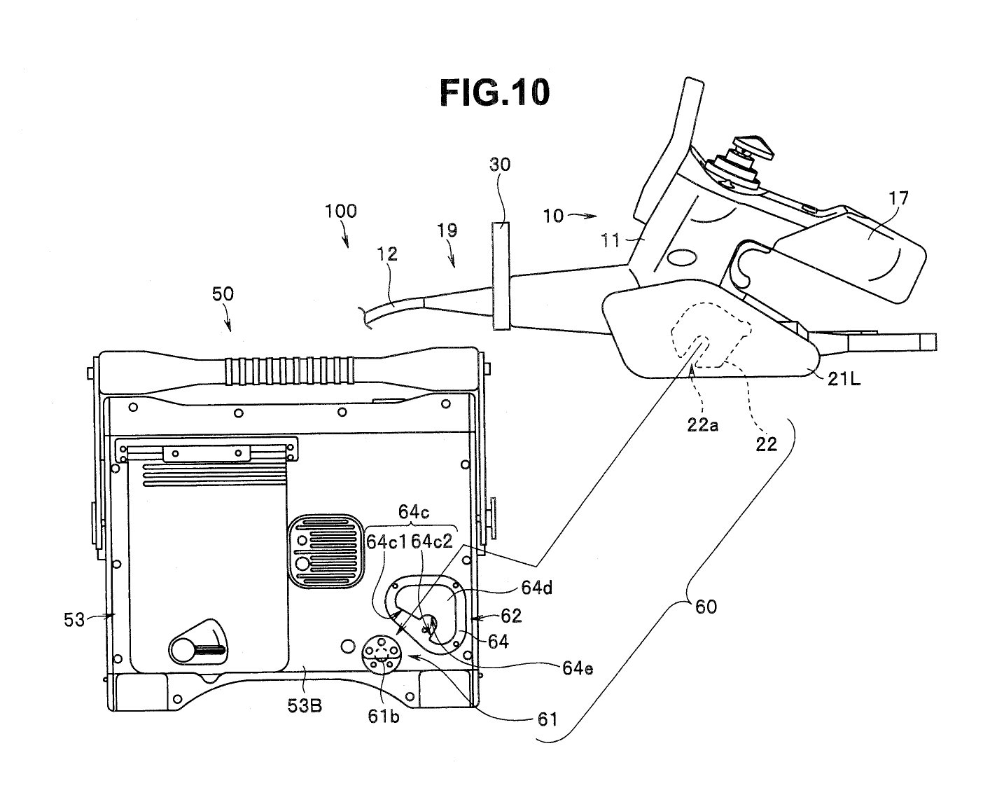

As shown in FIG. 3, a hanger 22 is provided, for example, on a side surface of a right leg portion 21R which is one of leg portions 21L, 21R of the operation portion 11 configuring the endoscope main body 10. The hanger 22 is a first attaching/detaching portion of the attaching/detaching mechanism (see the reference numeral 60 in FIG. 10), and configures an operation-portion-side attaching/detaching portion.

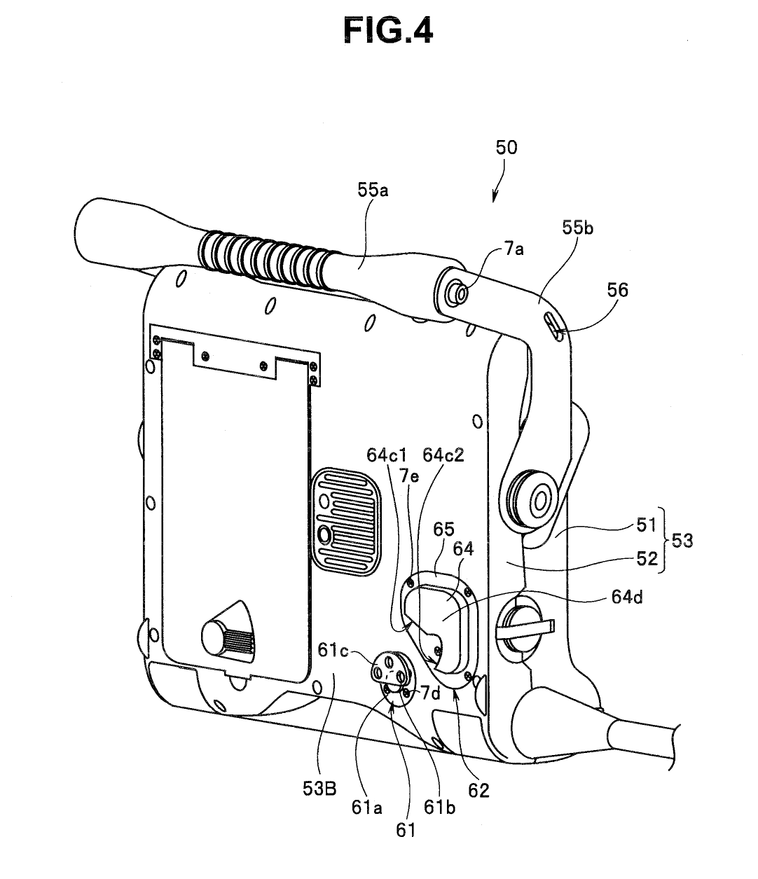

On the other hand, as shown in FIG. 4, a main-body-side attaching/detaching portion 61 as a second attaching/detaching portion of the attaching/detaching mechanism 60 is provided on a back surface 53B of the exterior portion 53 configuring the apparatus main body 50. The hanger 22 is detachably attached to the main-body-side attaching/detaching portion 61. The reference numeral 62 denotes a stopper portion. The stopper portion 62 is an operation portion-cum-holding portion which serves both as a falling-off preventing portion and a switching operation portion.

The hanger 22 shown by the two-dot chain lines in FIG. 3 is formed by a rigid resin member, for example. The hanger 22 includes a protruding portion 22b and a fixing portion 22e. The protruding portion 22b has an engaging groove 22a. The fixing portion 22e has a holding portion 22d. The holding portion 22d includes holding surfaces 22d1 and 22d2 which are perpendicular to each other, and a curved surface convex portion 22d3, for example. A pair of bosses 22c are protrudingly arranged on the rear surface of the protruding portion 22b, sandwiching the engaging groove 22a therebetween.

The hanger 22 is fixed using a screw 7c, with the rear side surface of the fixing portion 22e and the rear surfaces of the bosses 22c brought into contact with the side surface of the right leg portion 21R. The reference numeral 22f denotes a terminal end of the engaging groove 22a.

As shown in FIG. 4, the main-body-side attaching/detaching portion 61 is a hanger engaging portion. The main-body-side attaching/detaching portion 61 is made of metal such as stainless steel. The main-body-side attaching/detaching portion 61 includes a fixing disk portion 61a, a supporting pole 61b, and a holding portion 61c. The supporting pole 61b is disposed to stand from a center portion of the fixing disk portion 61a. The holding portion 61c faces the fixing disk portion 61a across the supporting pole 61b. The fixing disk portion 61a is fixed using a screw 7d.

The diameter dimension of the supporting pole 61b is set in consideration of the width dimension of the engaging groove 22a of the hanger 22. Specifically, the diameter dimension of the supporting pole 61b is so set as to be smaller than the width dimension of the engaging groove 22a by a preset dimension. The gap between the fixing disk portion 61a and the holding portion 61c is set in consideration of the thickness dimension of the protruding portion 22b of the hanger 22 so as to be larger than the thickness dimension of the protruding portion 22b by a preset dimension.

Note that the width dimension of the holding portion 61c is so formed as to be wider than at least the width dimension of the engaging groove 22a by a preset dimension.

Therefore, when the operator makes an aperture of the engaging groove 22a of the hanger 22 face the supporting pole 61b of the main-body-side attaching/detaching portion 61 and then brings the hanger 22 closer to the main-body-side attaching/detaching portion 61, the supporting pole 61b of the main-body-side attaching/detaching portion 61 is arranged in the engaging groove 22a of the hanger 22.

The protruding portion 22b of the hanger 22 is disposed in the gap of the main-body-side attaching/detaching portion 61, resulting in the state where the hanger 22 is hooked on, in other words, engaged with the main-body-side attaching/detaching portion 61.

The stopper portion 62 includes a coil spring as a biasing member not shown, a stopper 64, and a stopper regulation plate 65. The coil spring is a pressing spring and presses the stopper 64 such that the stopper 64 protrudes from the back surface 53B. The stopper 64 has a substantially convex-shaped cross section, and is made of rigid resin member or a metal member such as stainless steel. The stopper regulation plate 65 is configured of a thin plate made of metal such as stainless steel, and fixed on the back surface 53b with a screw 7e. According to such a configuration, an edge portion, not shown, of the stopper 64 comes into contact with the stopper regulation plate 65, which regulates a protruding amount of the stopper 64.

The stopper 64 configuring the stopper portion 62 is pushed up by a biasing force of the coil spring and arranged protruding from the back surface 53B. The stopper 64 protruding from the back surface 53B is depressed by pressing a pressing surface 64d against the biasing force of the coil spring.

In the present embodiment, the stopper 64 is protrudable and retractable. The pressing surface 64d of the stopper 64 is so set as to be switched between a protruding state where the stopper 64 protrudes by a predetermined amount with respect to the back surface 53B and a retracted state where the surface of the stopper 64 substantially agrees with the back surface 53B. When the stopper 64 is in the protruding state, the endoscope main body 10 can be held at the apparatus main body 50. On the other hand, when the stopper 64 is in the retracted state, the endoscope main body 10 is detachable from the apparatus main body 50.

When the hanger 22 is attached to the main-body-side attaching/detaching portion 61 in a predetermined attaching state, the stopper 64 is protruded by the biasing force of the coil spring. Then, contact surfaces 64c1, 64c2 of the stopper 64 come into contact with the holding surfaces 22d1, 22d2 of the hanger 22, and thereby the hanger is brought into a held state. In the held state, the hanger 22 engaged with the main-body-side attaching/detaching portion 61 is surely prevented from falling off.

Here, description will be made on working of the endoscope apparatus 1 including the insertion portion holder 30 and the attaching/detaching mechanism 60. Note that the insertion portion 12 of the endoscope apparatus main body 10 which is to be used is the insertion portion having the second diameter, that is, the diameter of the insertion portion is smaller than that of the insertion portion having the first diameter.

First, the working of the insertion portion holder 30 will be described.

As shown in FIG. 3, an operator arranges in advance the first through hole 31 of the insertion portion holder 30 at the base portion 19a of the break prevention member 19. Then, the insertion portion holder 30 is retained integrally with the operation portion 11 by the elastic force of the insertion portion holder 30.

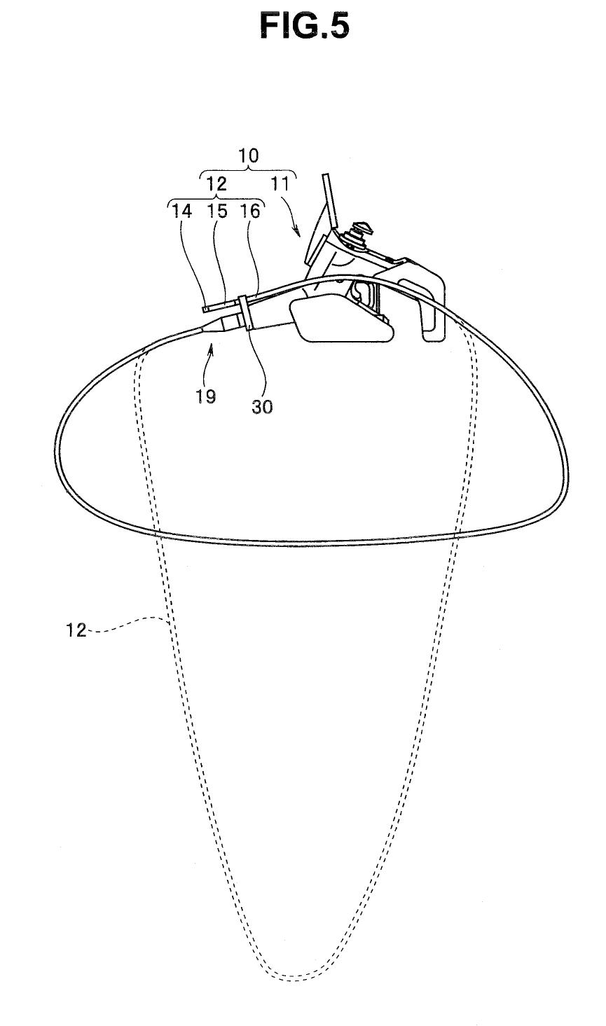

When the operator takes a quick break during examination, for example, the operator arranges the distal end side of the flexible tube portion 16 of the insertion portion 12 in the second holding portion 32b of the second through hole 32, as shown in FIG. 5. Then, the distal end side part of the insertion portion 12 is held by the second holding portion 32b by the elastic force of the insertion portion holder 30, which brings the insertion portion into a state of floating from a table surface, a floor surface, or the like.

The insertion portion holder is thus integrally attached to the operation portion, and the insertion portion is arranged in the holding portion. Then, the distal end side part of the insertion portion is held integrally with the operation portion through the insertion portion holder, which prevents an object from being placed on the distal end side part of the insertion portion, and also prevents the distal end side part from being stepped on by the foot.

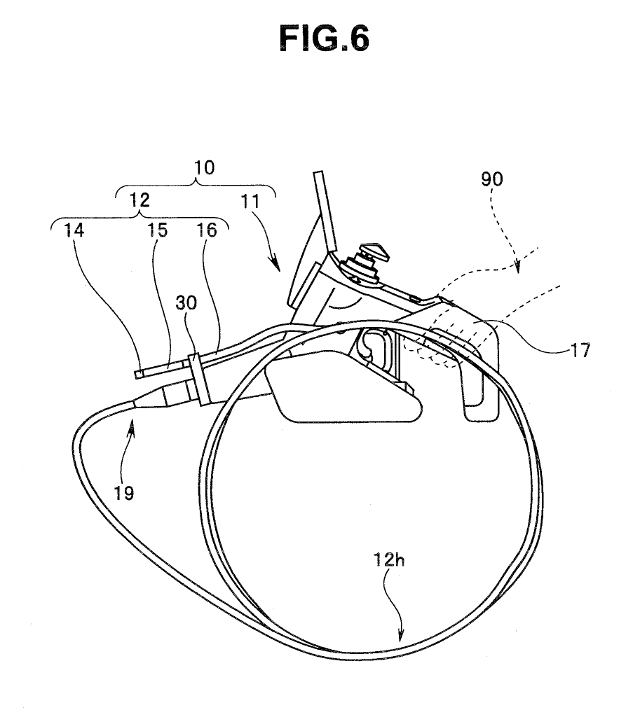

However, when the operation portion 11 is lifted up in the above-described held state, although the distal end side part of the insertion portion 12 is held integrally with the operation portion 11, the halfway portion of the insertion portion hangs long from the operation portion 11, as shown by the dashed lines in FIG. 5. Accordingly, when the operator suspends the examination and moves, before arranging the distal end side of the flexible tube portion 16 in the second holding portion 32b, the operator forms a spiral-shaped insertion portion 12h by rolling up the insertion portion 12 into a spiral by using elasticity of the flexible tube portion 16, as shown in FIG. 6. After that, the operator arranges the distal end side of the flexible tube portion 16 which is the distal end side part of the insertion portion 12 in the second holding portion 32b of the second through hole 32.

As a result, the operator lifts up the operation portion 11 by grasping a part of the spiral-shaped insertion portion 12h together with the grasping portion 17 with the thumb and fingers 90 as shown by the dashed lines in FIG. 6, which prevents the insertion portion 12 from hanging long from the operation portion 11.

Furthermore, the distal end side of the flexible tube portion 16 of the insertion portion 12 is held by the second holding portion 32b. Accordingly, it is possible to prevent the distal end portion 14 and the bending portion 15 of the insertion portion 12 from bumping against a wall or an obstacle, during carrying of the apparatus. As a result, it is possible to prevent the image pickup device, an optical lens, and the like provided in the distal end portion 14 from being damaged and also prevent the bending pieces configuring the bending portion 15 from being deformed.

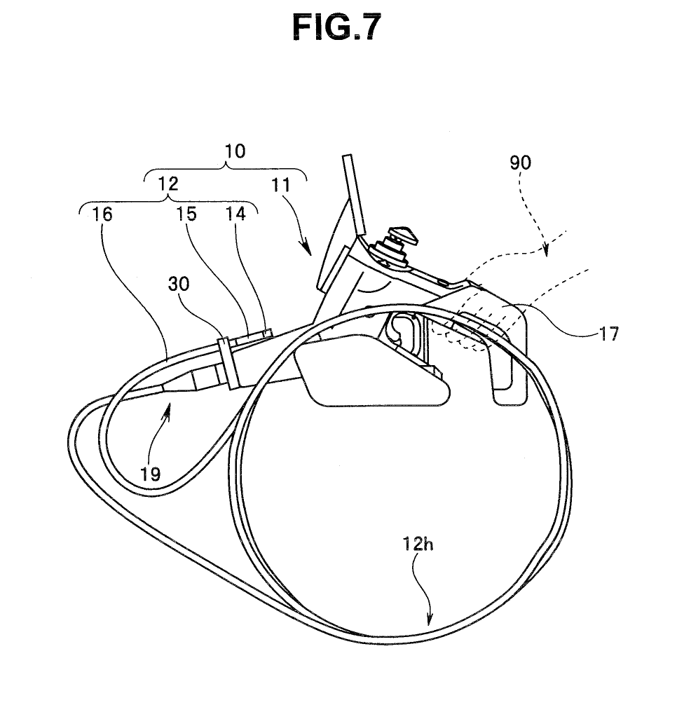

In the above-described FIG. 5 and FIG. 6, the distal end side part of the insertion portion 12 is protruded from the operation portion 11 side toward the insertion portion 12 side. However, when inserting and arranging the distal end side part of the insertion portion 12 in the second holding portion 32a, the operator may move the distal end portion 14 of the insertion portion 12 toward the direction of the operation portion 11. The operator may cause the second holding portion 32a to hold the insertion portion 12 such that the distal end portion 14 of the insertion portion 12 faces the direction of the grasping portion 17 of the operation portion 11, as shown in FIG. 7.

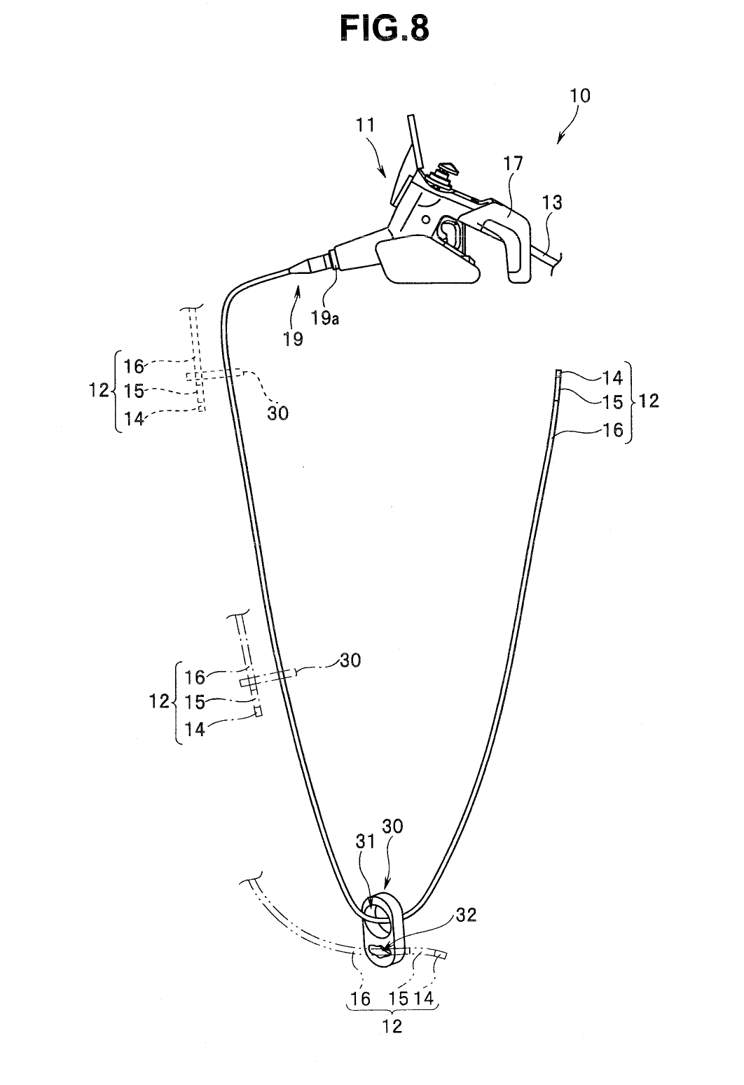

In addition, in the above-described FIG. 5 and FIG. 6, the insertion portion holder 30 is retained in advance at the base portion 19a of the break prevention member 19. However, as shown in FIG. 8, the insertion portion holder 30 may be arranged at the halfway position of the insertion portion 12.

In this case, the operator first causes the second holding portion 32b to hold the distal end side part of the insertion portion 12, as shown by the two-dot chain lines in FIG. 8. After that, the operator gradually moves the insertion portion holder 30 in the longitudinal direction toward the operation portion 11 side, as shown by one-dot chain lines and the dashed lines. Then, the operator finally arranges the first through hole 31 of the insertion portion holder 30 at the base portion 19a of the break prevention member 19, thereby allowing the insertion portion holder 30 to be retained integrally with the operation portion 11.

Accordingly, similarly as in the embodiment shown in FIG. 5, the insertion portion 12 can be held integrally with the operation portion 11 through the insertion portion holder 30.

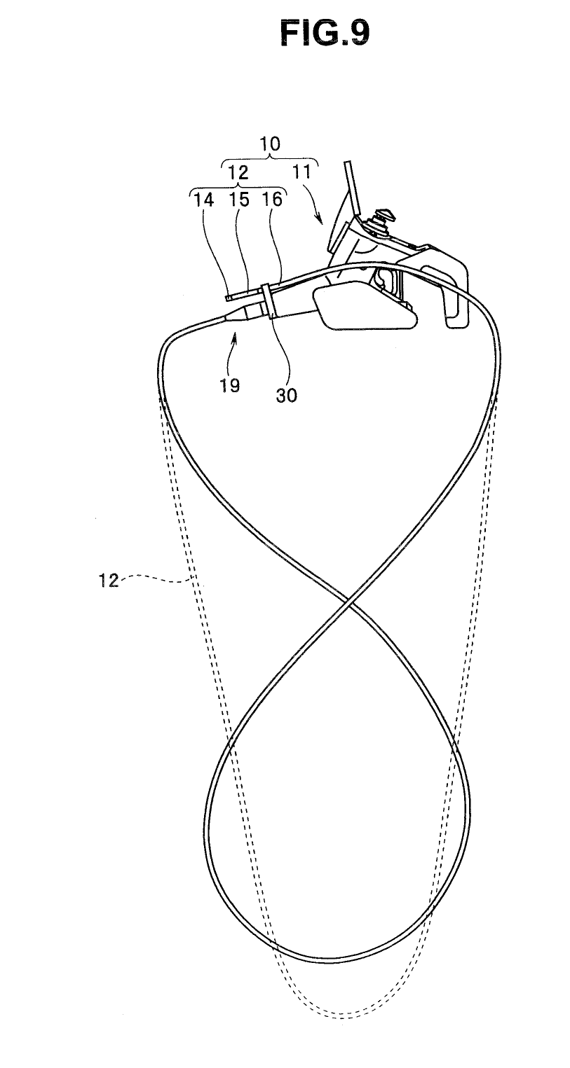

Furthermore, in the above-described FIG. 6, after forming the spiral-shaped insertion portion 12h by rolling up the insertion portion 12 into a spiral, the operator arranges the distal end side of the flexible tube portion 16 of the insertion portion 12 in the second holding portion 32b. However, as shown by the dashed lines in FIG. 9, after allowing the insertion portion 12 to be held integrally with the operation portion 11 through the insertion portion holder 30, the operator may form the spiral-shaped insertion portion 12h as shown in FIG. 6 by forming the insertion portion 12 in a figure of eight (shape of two rings) as shown in the solid lines in FIG. 9 and turning the lower ring portion up toward the operation portion 11, for example.

This makes it possible to solve such a problem that the insertion portion 12 hangs low from the operation portion 11, similarly as in the embodiment shown in FIG. 6.

Next, the working of the attaching/detaching mechanism 60 is described.

In the endoscope apparatus 1 of the present embodiment, the attaching/detaching mechanism 60 has both the function for easily attaching the endoscope main body 10 and the apparatus main body 50 integrally to each other and for detaching the endoscope main body 10 and the apparatus main body 50 from each other as separate bodies and the function as a winding portion holding portion for holding the insertion portion wound in a spiral shape.

As shown in FIG. 10, when arranging the operation portion 11 of the endoscope main body 10 on the apparatus main body 50, the operator first makes the engaging groove 22a of the hanger 22 shown by the dashed lines, which is provided at the operation portion 11, substantially face the main-body-side attaching/detaching portion 61 provided on the back surface 53B of the exterior portion 53. Then, the operator brings the hanger 22 closer to the stopper portion 62 separated from the main-body-side attaching/detaching portion 61.

Next, the operator checks whether the hanger 22 moved to a position where a front-side face of the hanger 22 substantially faces a front-side face of the stopper portion 62. After checking the front-side face of the hanger 22 and the front-side face of the stopper portion 62 have faced each other, the operator moves the front-side face of the hanger 22 toward the front-side face of the stopper portion 62. Then, the operator brings a front-side face of the protruding portion 22b into contact with the pressing surface 64d of the stopper 64. After that, the operator depresses the stopper 64 protruding from the back surface 53B against the biasing force of the coil spring until the stopper 64 is brought into a retracted state. When the depression of the stopper to reach the retracted state is completed, the operator moves the aperture of the engaging groove 22a of the hanger 22 toward the supporting pole 61b.

The operator performs position adjustment such that the supporting pole 61b is arranged in the engaging groove 22a, and arranges the supporting pole 61b in the engaging groove 22a. After that, the operator moves the hanger 22 until the terminal end 22f of the engaging groove 22a comes into contact with the supporting pole 61b. Then, the hanger 22 is engaged with the main-body-side attaching/detaching portion 61 in a predetermined state.

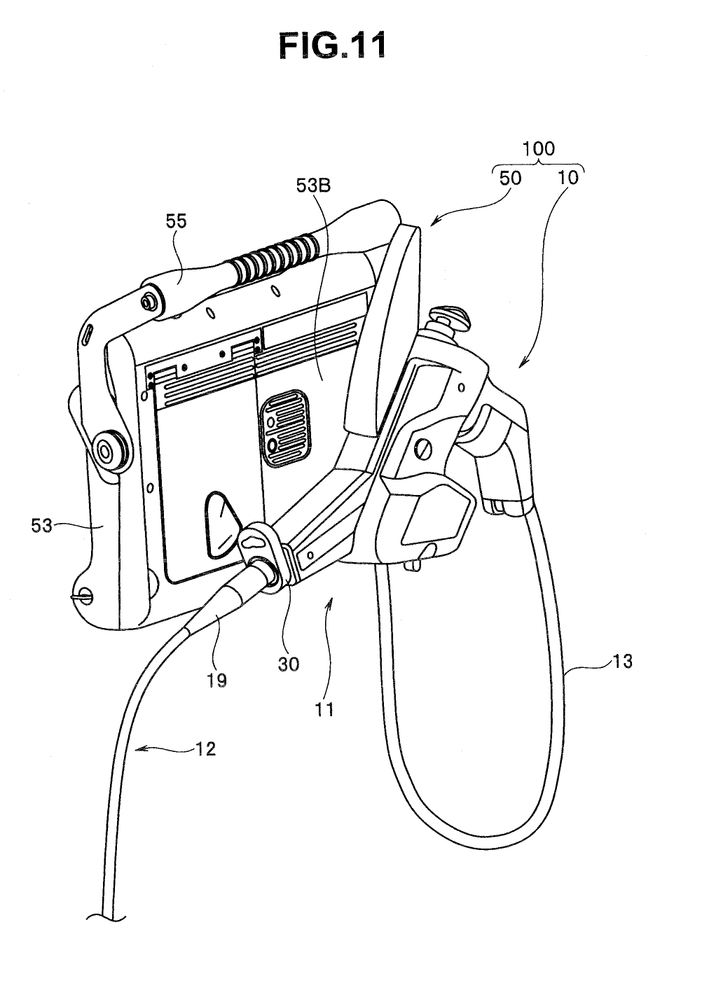

When the hanger 22 is brought into the predetermined engaged state, the pressing force against the stopper 64 by the hanger 22 is released. The stopper 64 is protruded by the biasing force of the coil spring. This allows the operation portion 11 to be integrally held with respect to the back surface 53B of the apparatus main body 50, as shown in FIG. 11. In the held state, the contact surfaces 64c1, 64c2 of the stopper 64 come into contact with the holding surfaces 22d1, 22d2 of the hanger 22, and thereby the operation portion 11 is attached to the exterior portion 53 while being prevented from rotating with respect to and falling off from the exterior portion 53.

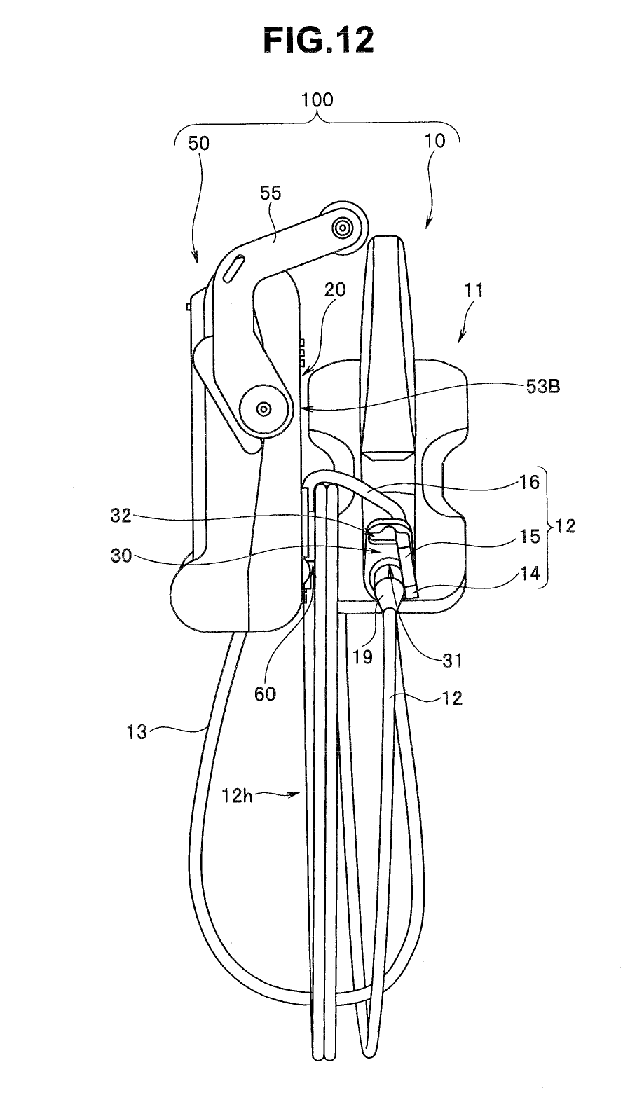

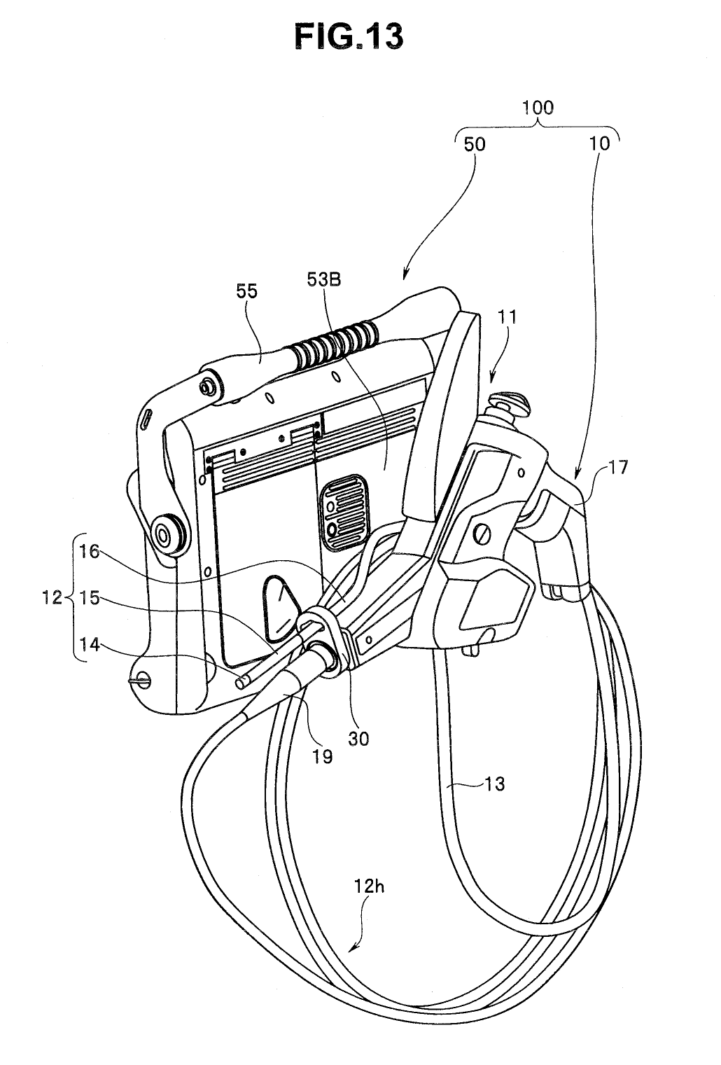

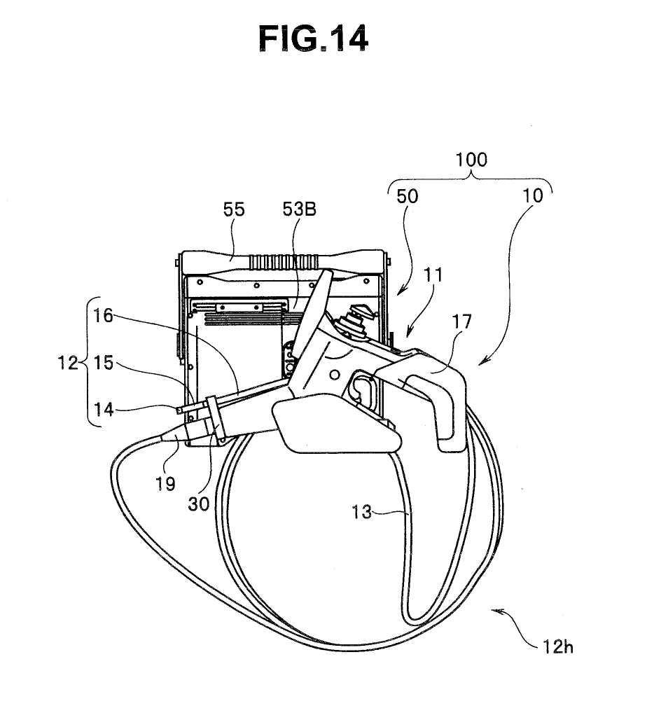

In the held state, the spiral-shaped insertion portion 12h rolled up and bundled in a spiral shape as shown in FIG. 6 is arranged on the attaching/detaching mechanism 60 serving also as the winding portion holding portion, through a gap 20 between the operation portion 11 and the back surface 53B shown in FIG. 12. As a result, as shown in FIGS. 12 to 14, the spiral-shaped insertion portion 12h is bundled and arranged between the operation portion 11 and the back surface 53B, and held in a hanging state on the operation portion 11 of the endoscope main body 10. In the hanging arrangement state, the spiral-shaped insertion portion 12h covers and wraps the attaching/detaching mechanism 60, which prevents the spiral shape of the spiral-shaped insertion portion 12h from deforming.

Thus, after the operation portion is integrally attached to the exterior portion of the apparatus main body through the attaching/detaching mechanism, the spiral-shaped portion is arranged between the operation portion and the back surface, and the distal end side part of the insertion portion is held in the second holding portion of the insertion portion holder. As a result, the spiral-shaped portion bundled in a spiral shape is held on the operation portion in a hanging state, so that it is possible to prevent the spiral shape of the spiral-shaped insertion portion from deforming

Therefore, by hanging the spiral-shaped insertion portion on the operation portion attached to the exterior portion, such a problem that the insertion portion hangs long from the operation portion is solved. In addition, the operator can carry the endoscope apparatus by grasping the handle, without grasping the spiral-shaped insertion portion.

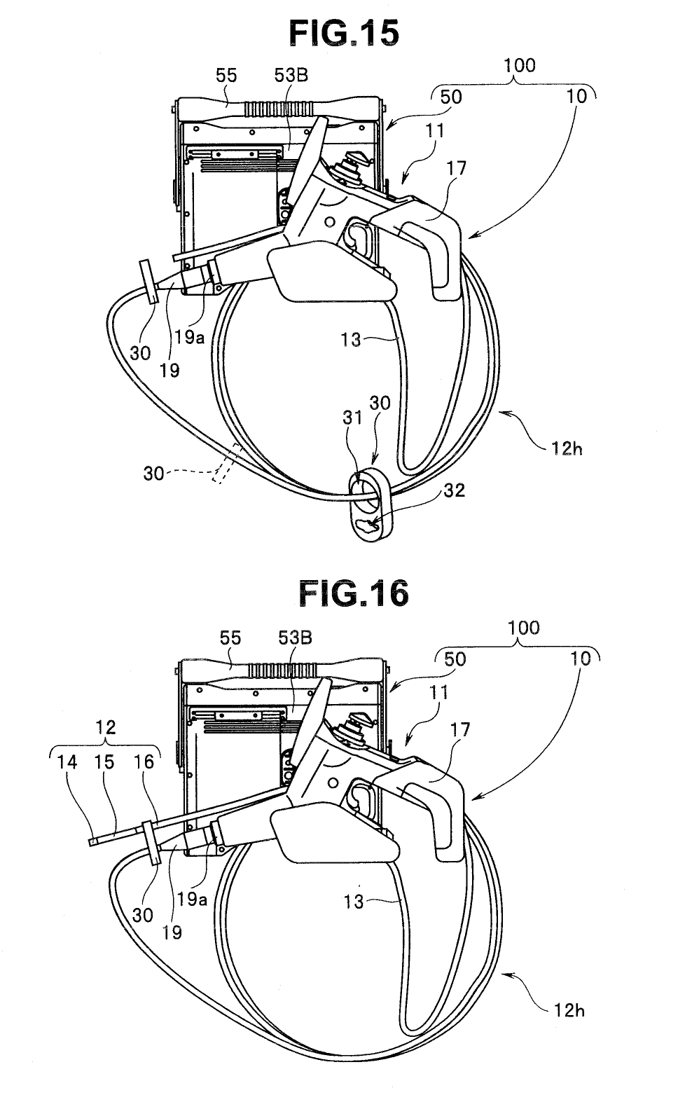

Note that, in the embodiment shown in FIGS. 12 to 14, the insertion portion holder 30 is retained in advance at the break prevention member 19 as shown in FIG. 6, the distal end side part of the insertion portion 12 is held in the second holding portion 32b of the insertion portion holder 30, and the insertion portion 12 includes at a part thereof the spiral-shaped insertion portion 12h bundled in a spiral shape. The spiral-shaped insertion portion 12h is arranged on the attaching/detaching mechanism 60 through the gap 20 between the operation portion 11 and the back surface 53B, which prevents the insertion portion 12 from hanging long from the operation portion 11. However, as shown in FIG. 15, if the insertion portion 12 is rolled up into a spiral with the insertion portion holder 30 arranged at the halfway position of the insertion portion 12, also the same working and effects as described above can be obtained.

That is, first, in a state where the operation portion 11 is attached to the exterior portion 53 while being prevented from rotating with respect to and falling off from the exterior portion 53, the operator passes the flexible tube portion 16 through the gap between the operation portion 11 and the back surface 53B repeatedly to form the spiral-shaped insertion portion 12h.

Next, the operator moves the insertion portion holder 30 arranged at the halfway position of the insertion portion 12 along the longitudinal axis direction to bring the insertion portion holder 30 close to the distal end portion 14 of the insertion portion 12.

Then, as shown in FIG. 16, the operator arranges the distal end side of the flexible tube portion 16 which is the distal end side part of the insertion portion 12 in the second holding portion 32b of the second through hole 32, without arranging the first through hole 31 of the insertion portion holder 30 at the base portion 19a of the break prevention member 19. At this time, the operator presses the pressing portion 35 with his or her fingers to obtain the second through hole 32D. The operator arranges the distal end side part of the insertion portion 12 in the vicinity of the second holding portion 32b. After that, the operator releases the pressing against the pressing portion 35. Then, the distal end side part of the insertion portion 12 is held by the second holding portion 32b.

As a result, similarly as in the above-described embodiment, the present embodiment solves such a problem that the insertion portion 12 hangs long from the operation portion 11, and allows the operator to carry the endoscope apparatus 1 by grasping the handle 55 without grasping the spiral-shaped insertion portion 12h.

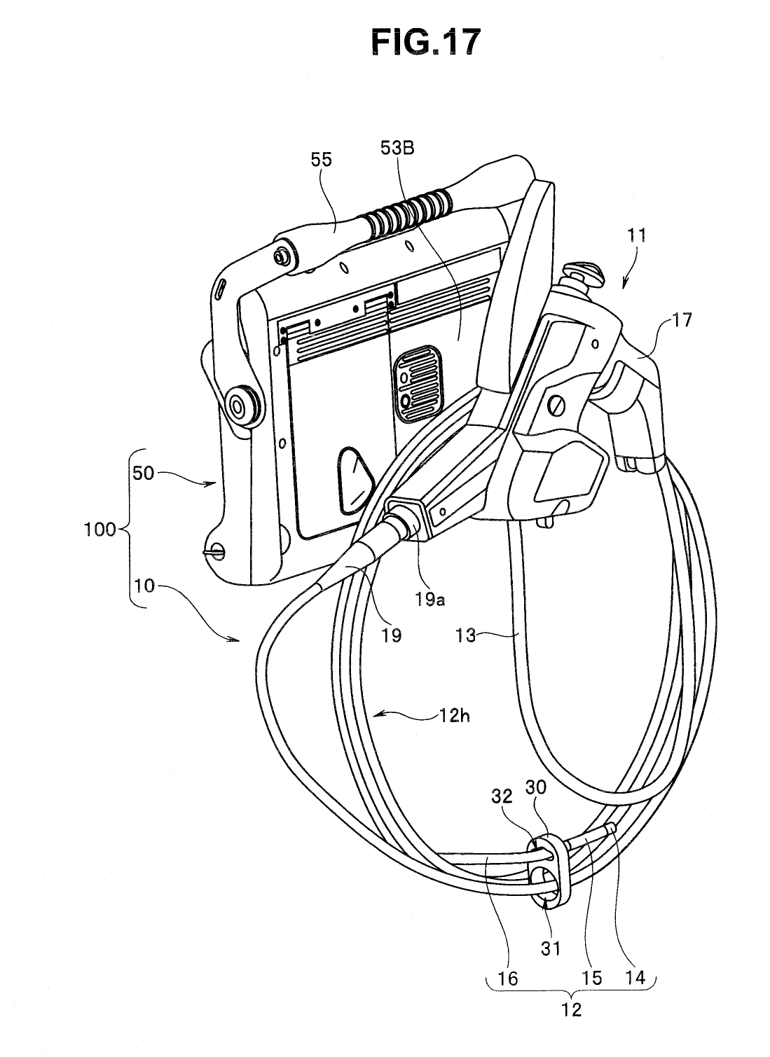

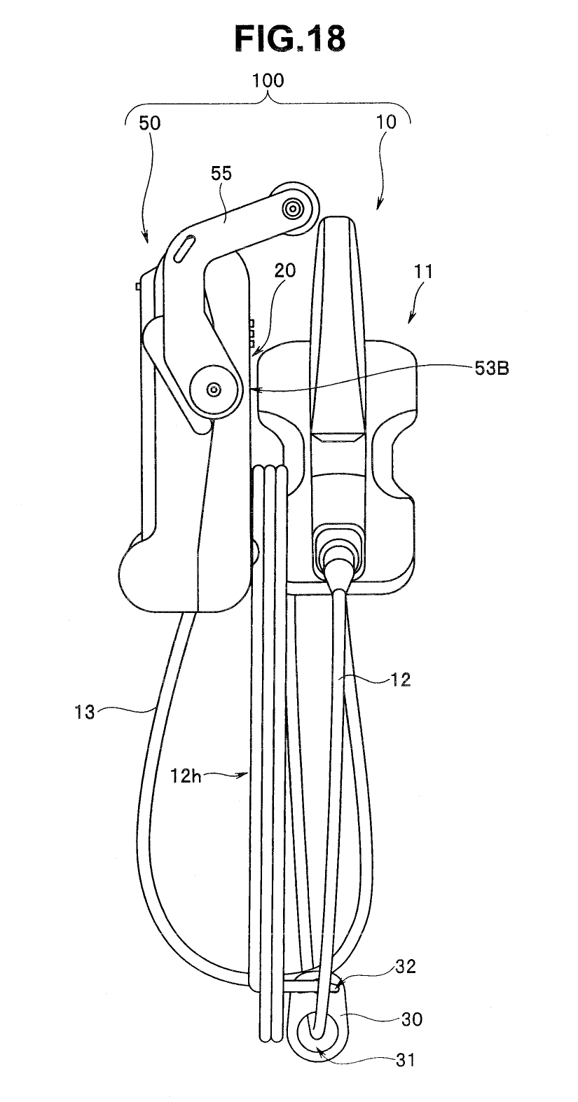

In addition, in the embodiment shown in FIGS. 15 and 16, the operator moves the insertion portion holder 30 arranged at the halfway position of the insertion portion 12 along the longitudinal axis direction, to bring the insertion portion holder 30 closer to the distal end portion 14 of the insertion portion 12. However, when the operator formed the spiral-shaped insertion portion 12h by repeatedly passing the flexible tube portion 16 through the gap between the operation portion 11 and back surface 53B, as described above, the distal end portion 14 of the insertion portion 12 is arranged in the vicinity of the insertion portion holder 30 in some cases, as shown in FIGS. 17 and 18.

In this case, the operator turns the insertion portion holder 30 upside down, for example, to place the distal end side part of the insertion portion 12 in the second holding portion 32b.

As a result, as in the above-described embodiment, the present embodiment solves such a problem that the insertion portion 12 hangs long from the operation portion 11, and allows the operator to carry the endoscope apparatus 1 by grasping the handle 55 without grasping the spiral-shaped insertion portion 12h.

In the above-described embodiment, the insertion portion 12 is passed through the first through hole 31 of the insertion portion holder 30 in advance. In other words, the first through hole 31 is a hole through which the insertion portion 12 is passed first. However, when the insertion portion 12 of the endoscope main body 10 which is to be used is the insertion portion having the second diameter, the spiral-shaped insertion portion 12h may be formed as described below.

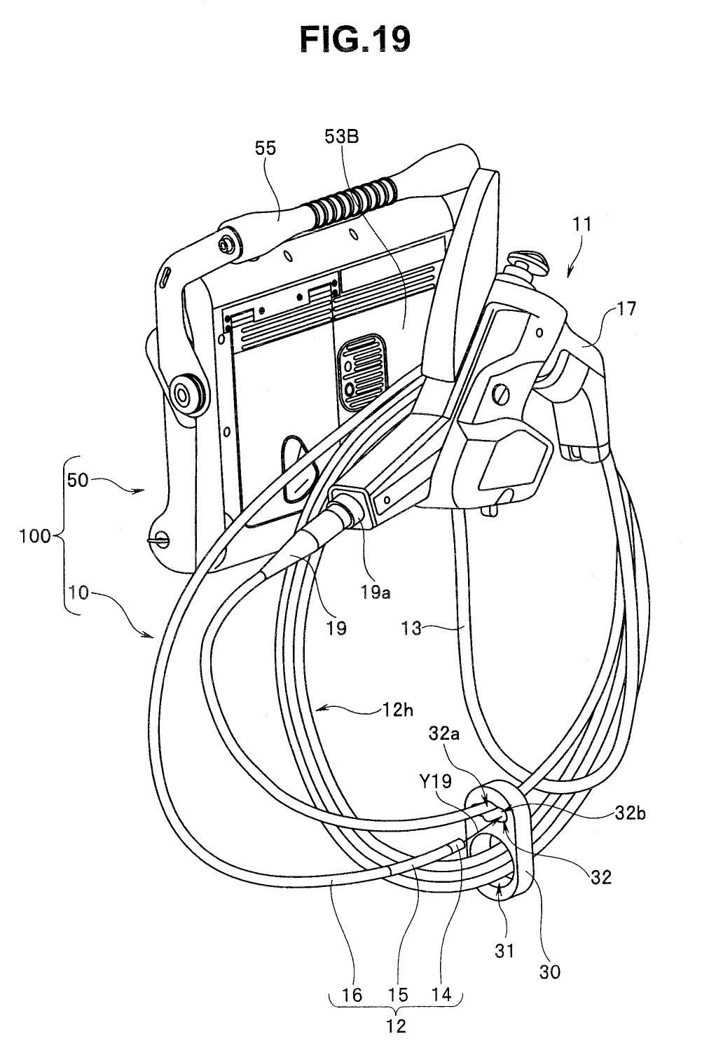

That is, as shown in FIG. 19, the operator first passes the insertion portion 12 having the second diameter through the first holding portion 32a of the second through hole 32 of the insertion portion holder 30. Next, the operator passes the flexible tube portion 16 through the gap between the operation portion 11 and the back surface 53B. After that, the operator passes the insertion portion 12 through the first through hole 31 of the insertion portion holder 30. Next, the operator repeatedly performs the operation of passing the flexible tube portion 16 through the gap between the operation portion 11 and the back surface 53B and the operation of passing the insertion portion 12 through the first through hole 31. The operator finally arranges the distal end side part of the insertion portion 12 toward the second holding portion 32b of the second through hole 32 of the insertion portion holder 30, as shown by an arrow Y19.

As a result, the spiral-shaped insertion portion 12h passing through the first through hole 31 is formed.

The insertion portion 12 is thus passed through between the operation portion 11 and the back surface 53B several times and also passed through the first through hole 31 several times, to form the spiral-shaped insertion portion 12h, thereby surely preventing the spiral-shaped insertion portion 12h from being separated during carrying of the endoscope apparatus.

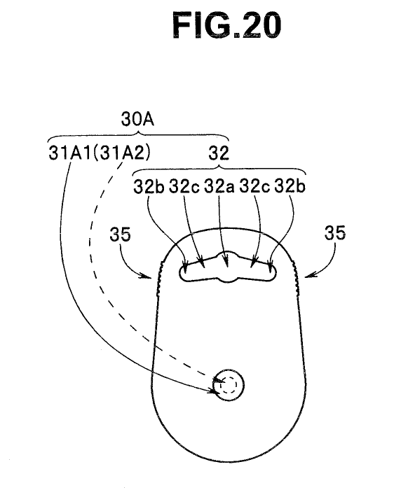

In addition, in the above-described embodiment, the first through hole 31 of the insertion portion holder 30 is substantially a circular shape, and is so formed as to have an inner diameter larger than the outer diameter of the insertion portion 12 and detachably retained by the elastic force at the base portion 19a of the break prevention member 19 configuring the operation portion 11, for example. However, the configuration of the first through hole 31 is not limited to the above-described configuration. As shown in FIG. 20, an insertion portion holder 30A may be retained with respect to the insertion portion 12 by an elastic force.

As shown in FIG. 20, the insertion portion holder 30A includes a first through hole 31A and a second through hole 32. The hole diameter of the first through hole 31A is set based on the first diameter or the second diameter. That is, the insertion portion holder 30A is configured by a holder of a type including the second through hole 32 and the first through hole 31A1, shown by the solid line, which corresponds to the diameter dimension of the insertion portion having the first diameter, or a holder of a type including the second through hole 32 and a first through hole 31A2, shown by the dashed line, which corresponds to the diameter dimension of the insertion portion having the second diameter.

When the insertion portion corresponding to the first through hole 31A1 or 31A2 of the insertion portion holder 30A is inserted through the corresponding through hole, the insertion portion is held in the first through hole 31A1 or 31A2 by the elastic force of the holder 30A.

The elastic force of the holder 30A, i.e., the force for holding the insertion portion is set such that the operator can move the insertion portion 12 in the longitudinal direction against the elastic force and rotate the insertion portion 12 around the longitudinal axis. Therefore, the insertion portion holder 30A can be arranged at an arbitrary position between the distal end and the proximal end of the insertion portion 12. Other configurations are the same as those of the insertion portion holder 30. The same components are attached with the same reference numerals and descriptions thereof will be omitted.

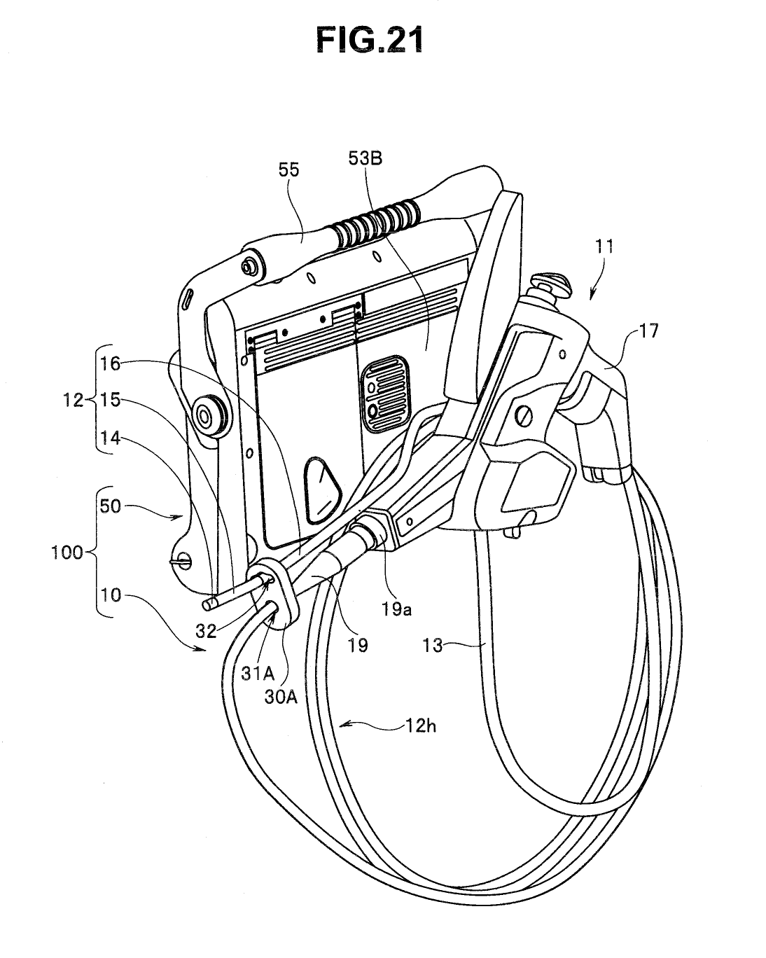

According to the above-described configuration, the insertion portion holder 30A is allowed to be retained at an arbitrary position of the insertion portion 12 and the distal end side part of the insertion portion 12 is allowed to be held in the holding portion 32a or 32b of the insertion portion holder 30A, as shown in FIG. 21.

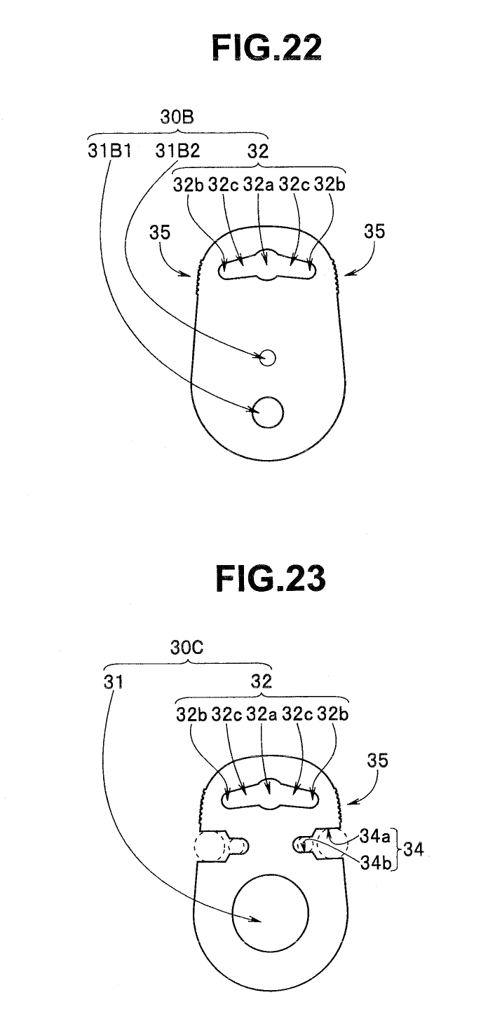

As shown in FIG. 22, an insertion portion holder 30B may be configured instead of the insertion portion holder 30A. The insertion portion holder 30B includes, in addition to the second through hole 32a, a first through hole 31B1 for a first insertion portion which corresponds to the insertion portion having the first diameter and a first through hole 31B2 for a second insertion portion which corresponds to the insertion portion having the second diameter. Other configurations are the same as those of the insertion portion holders 30, 30A. The same components are attached with the same reference numerals and descriptions thereof will be omitted.

According to such a configuration, one insertion portion holder 30B is applicable to both the insertion portion having the first diameter and the insertion portion having the second diameter. Therefore, there is no need for forming a plurality of kinds of insertion portion holders 30A in accordance with the diameters of the insertion portions.

In addition, as shown in FIG. 23, instead of the insertion portion holders 30, 30A, and 30B, a pair of holding grooves 34 for holding the insertion portion may be formed on an outer peripheral surface of an insertion portion holder 30C, for example. Each of the holding grooves 34 includes, on the opening side of the groove, a first groove 34a for pressing and holding the insertion portion having the first diameter and, on the bottom side of the groove, a second groove 34b for pressing and holding the insertion portion having the second diameter. Other configurations are the same as those of the insertion portion holders 30, 30A and 30B. The same components are attached with the same reference numerals and descriptions thereof will be omitted.

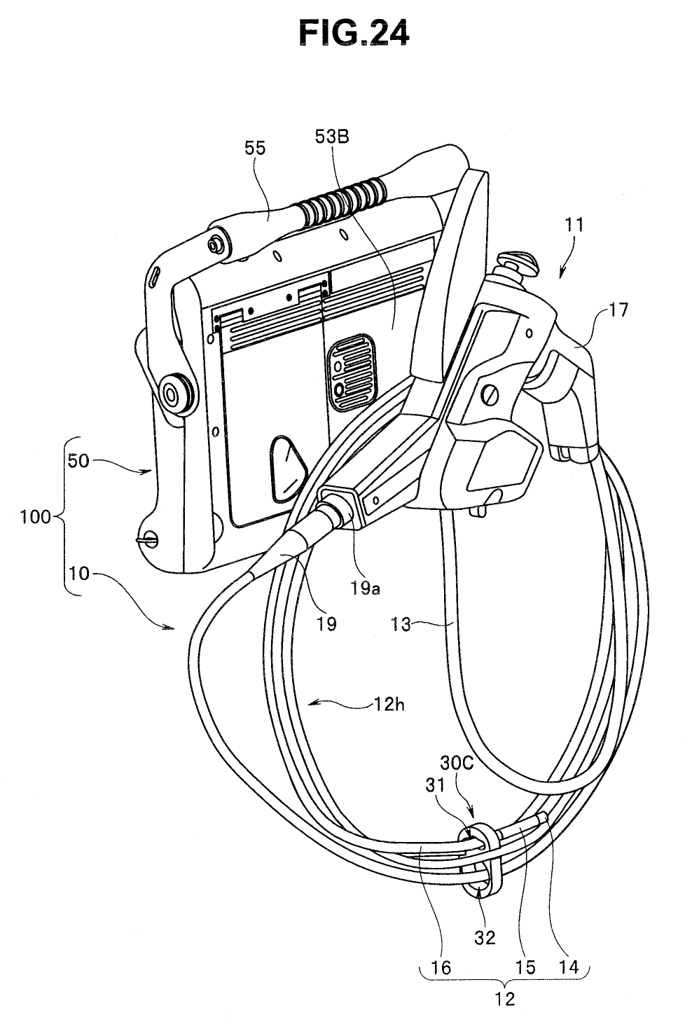

According to such a configuration, as shown in FIG. 24, the insertion portion 12 is passed through between the operation portion 11 and the back surface 53B several times and the insertion portion 12 is compressedly arranged in the pair of holding grooves 34 at least twice, thereby forming the spiral-shaped insertion portion 12h. This configuration surely prevents the spiral-shaped insertion portion 12h from being separated during the carrying of the endoscope apparatus.

Now, description will be made on a procedure of detaching the operation portion 11 from the exterior portion 53.

When separating the endoscope main body 10, the operation portion 11 of which is attached on the back surface 53B of the apparatus main body 50, the operator presses the pressing surface 64d of the stopper 64 with his or her thumbs and fingers 90 grasping the grasping portion 17, for example. Then the operator depresses the stopper 64 protruding from the back surface 53b against the biasing force of the coil spring until the stopper is brought into the retracted state. After that, the operator moves the hanger 22 attached to the main-body-side attaching/detaching portion 61 toward the direction of the stopper portion 62.

Since the stopper 64 is depressed to the retracted state, the front-side face of the hanger 22 is arranged on the pressing surface 64d of the stopper 64. At this time, the operator moves the hanger 22 away from the main-body-side attaching/detaching portion 61. Then, the engaged state between the main-body-side attaching/detaching portion 61 and the hanger 22 is released, and the endoscope main body 10 and the apparatus main body 50 are separated from each other again.

Incidentally, also in the above-described embodiments shown in FIGS. 5 to 9, the spiral-shaped insertion portion 12h is desired to be bundled as shown in the embodiments in FIGS. 12 to 24.

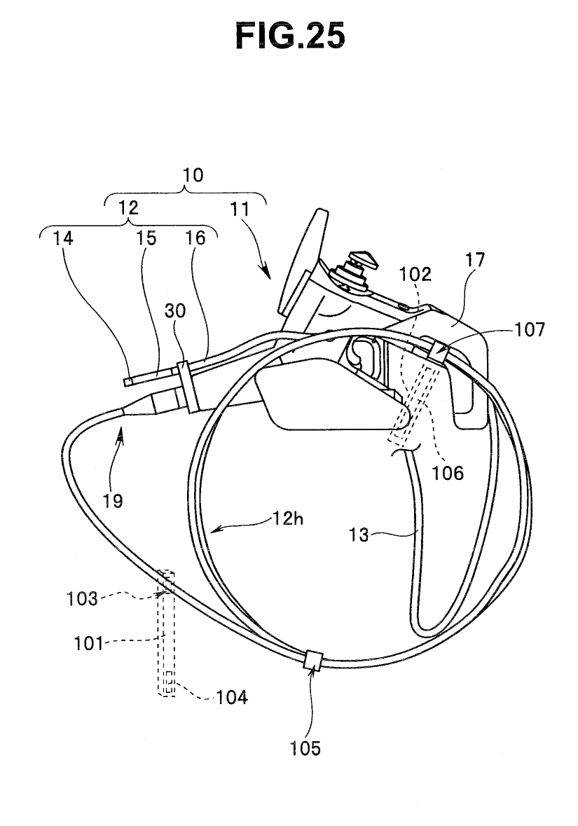

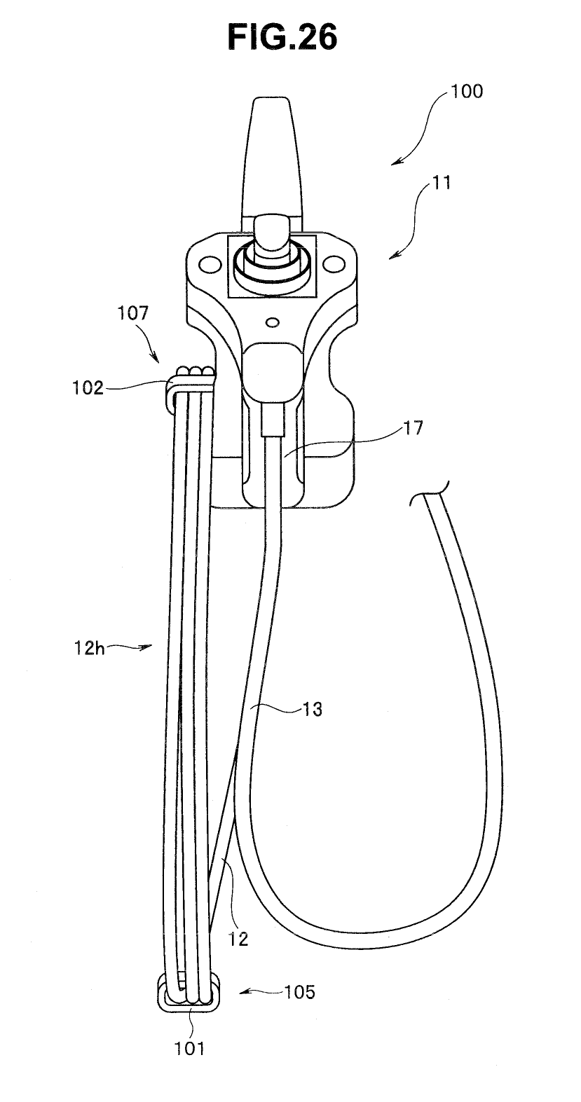

Therefore, the embodiment shown in FIG. 25 and FIG. 26 includes, as winding portion holding portions, a first tying band 101 which is a tying member arranged on the insertion portion 12, and a second tying band 102 which is a tying member arranged at the operation portion 11.

As shown in FIG. 25, the first tying band 101 is a strap and includes a through hole 103 on one end side thereof and a first hook and loop fastener 104, for example, as detachable tying means.

The through hole 103 is a hole through which the insertion portion 12 can be passed. The first tying band 101 is movable in the longitudinal axis direction and rotatable around the longitudinal axis, in the state where the insertion portion 12 is passed through the through hole 103.

One side (hook side, for example) of the first hook and loop fastener 104 is provided on one surface side of the first tying band 101 so as to be located on an end portion distal from the through hole 103. The other side (loop side, for example, not shown) of the first hook and loop fastener 104 is provided on the other surface side of the first tying band 101 so as to be located on an end portion near to the through hole 103. The one side of the first hook and loop fastener 104 on the one surface side of the first tying band 101 is arranged on the other side of the hook and loop fastener 104 on the other surface side of the first tying band 101, and thereby the first tying band 101 forms a first strap loop 105.

The second tying band 102 is also a strap. One end side of the second tying band 102 is fixed to the grasping portion 17 of the operation portion 11 with adhesive, for example. One side (hook side, for example) of a second hook and loop fastener 106 is provided on one surface side of the second tying band 102 so as to be located at an end portion on the lower side in the drawing. The other side (loop side, for example, not shown) of the second hook and loop fastener 106 is provided on the other surface side of the second tying band 102. The second tying band 102 becomes a second strap loop 107 similarly as the first tying band 101, by arranging the one side of the second hook and loop fastener 106 located on the one surface side of the second tying band 102 on the other side (not shown) of the second hook and loop fastener 106 located on the other surface side of the second tying member 102.

Here, description will be made specifically on the working of the tying band. When suspending the examination and moving, the operator first rolls up the insertion portion into a spiral to form the spiral-shaped insertion portion 12h. Next, the operator arranges the distal end side of the flexible tube portion 16 which is the distal end side part of the insertion portion 12 in the second holding portion 32b of the second through hole 32. After that, the operator brings the first tying band 101 and the second tying band 102 into a banded state (the hook side and the loop side of the hook and loop fastener are put together), respectively, in this order or in the reverse order.

When bringing the first tying band 101 into the banded state, the operator moves the first tying band 101 in the longitudinal axis direction and arranges the first tying band at the lower side in the drawing of the spiral-shaped insertion portion 12h. After that, in order to bundle the spiral-shaped insertion portion 12h together, the operator wraps the first tying band 101 on the spiral-shaped insertion portion 12h so as to pass across the longitudinal axis. Finally, the operator puts the hook side and the loop side of the first hook and loop fastener 104 together. As a result, the spiral-shaped insertion portion 12h is bundled together with the first tying band 101, as shown in FIGS. 25 and 26.

On the other hand, when bringing the second tying band 102 into the banded state, the operator arranges a desired portion on the upper side in the drawing of the spiral-shaped insertion portion 12h in the vicinity of the grasping portion 17. After that, in order to bundle the spiral-shaped insertion portion 12h together, the operator wraps the second tying band 102 on the spiral-shaped insertion portion 12h similarly as described above. Finally, the operator puts the hook side and the loop side of the second hook and loop fastener 106 together. As a result, as shown in FIG. 25 and FIG. 26, the spiral-shaped insertion portion 12h is bundled together with the second tying band 102 and attached integrally to the operation portion 11 in the bundled state.

According to such a configuration, it is possible to solve such a problem that the insertion portion 12 hangs long from the operation portion 11. In addition, the operator can carry the endoscope apparatus 1 without grasping the spiral-shaped insertion portion 12h.

In the present embodiment, the second tying band 102 is fixed to the grasping portion 17. However, the fixing position is not limited to the grasping portion 17, and may be at the leg portions 21R, 21L, or the like.

Furthermore, also in an endoscope apparatus 100A shown in FIG. 27, the same working and effects as described above can be obtained by providing the first tying band 101 to the insertion portion 12 and the second tying band 102 to the operation portion 11A.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An insertion portion holder comprising,
a plane portion formed in a flat shape with an elastic member, the plane portion including:
a first through hole formed on the plane portion, for passing therethrough an insertion portion having flexibility extended from an endoscope operation portion; and
a second through hole formed on the plane portion adjacently to the first through hole, for holding a distal end side part of the insertion portion.

2. The insertion portion holder according to claim 1, wherein a shape of the first through hole is different from a shape of the second through hole.

3. The insertion portion holder according to claim 2, wherein the second through hole has a long hole shape and includes at least:
a first insertion portion holding portion for holding, by an elastic force, a distal end side part of an insertion portion having a first diameter; and
a second insertion portion holding portion for holding, by an elastic force, a distal end side part of an insertion portion having a second diameter whose diameter is smaller than that of the insertion portion having the first diameter.

4. The insertion portion holder according to claim 2, wherein the first through hole is a hole through which the insertion portion is passed through first, and the first through hole has a hole shape configured such that the insertion portion holder is movable in a longitudinal axis direction of the insertion portion and rotatable around the longitudinal axis.

5. The insertion portion holder according to claim 4, wherein the first through hole is configured such that the insertion portion holder is detachably retained with respect to the endoscope operation portion by an elastic force.

6. The insertion portion holder according to claim 4, wherein the first through hole is configured such that the insertion portion holder can be retained with respect to the insertion portion by an elastic force.

7. The insertion portion holder according to claim 5, further comprising
a pressing portion for expanding a diameter of the first insertion portion holding portion and a diameter of the second insertion portion holding portion of the second through hole by being pressed, the pressing portion being formed on an outer peripheral surface of the plane portion.

8. The insertion portion holder according to claim 6, further comprising
a pressing portion for expanding a diameter of the first insertion portion holding portion and a diameter of the second insertion portion holding portion of the second through hole by being pressed, the pressing portion being formed on an outer peripheral surface of the plane portion.

9. The insertion portion holder according to claim 7, further comprising
a recessed portion for holding a halfway portion of the insertion portion by an elastic force, which is provided on the outer peripheral surface of the plane portion.

10. The insertion portion holder according to claim 8, further comprising
a recessed portion for holding a halfway portion of the insertion portion by an elastic force, which is provided on the outer peripheral surface of the plane portion.

11. An endoscope apparatus comprising:
an elongated endoscope insertion portion having on a distal end side thereof an illumination portion and an observation portion;
a first housing portion to which a proximal end portion of the endoscope insertion portion is connected;
a second housing portion electrically connected to the first housing portion; and
an insertion portion holder including a plane portion formed in a flat shape with an elastic member, a first through hole formed on the plane portion, for passing therethrough the insertion portion having flexibility extended from an endoscope operation portion, and a second through hole formed on the plane portion adjacently to the first through hole, for holding a distal end side part of the insertion portion.

12. The endoscope apparatus according to claim 11, wherein a shape of the first through hole is different from a shape of the second through hole.

13. The endoscope apparatus according to claim 12, wherein the second through hole has a long hole shape and includes at least:
a first insertion portion holding portion for holding, by an elastic force, a distal end side part of an insertion portion having a first diameter; and
a second insertion portion holding portion for holding, by an elastic force, a distal end side part of an insertion portion having a second diameter whose diameter is smaller than that of the insertion portion having the first diameter.

14. The endoscope apparatus according to claim 12, wherein the first through hole is a hole through which the insertion portion is passed through first, and the first through hole has a hole shape configured such that the insertion portion holder is movable in a longitudinal axis direction of the insertion portion and rotatable around the longitudinal axis.

15. The endoscope apparatus according to claim 14, wherein the first through hole is configured such that the insertion portion holder is detachably retained with respect to the endoscope operation portion by an elastic force.

16. The endoscope apparatus according to claim 14, wherein the first through hole is configured such that the insertion portion holder can be retained with respect to the insertion portion by an elastic force.

17. The endoscope apparatus according to claim 15, further comprising
a pressing portion for expanding a diameter of the first insertion portion holding portion and a diameter of the second insertion portion holding portion of the second through hole by being pressed, the pressing portion being formed on an outer peripheral surface of the plane portion.

18. The endoscope apparatus according to claim 16, further comprising
a pressing portion for expanding a diameter of the first insertion portion holding portion and a diameter of the second insertion portion holding portion of the second through hole by being pressed, the pressing portion being formed on an outer peripheral surface of the plane portion.

19. The endoscope apparatus according to claim 17, further comprising
a recessed portion for holding a halfway portion of the insertion portion by an elastic force, which is provided on the outer peripheral surface of the plane portion.

20. The endoscope apparatus according to claim 18, further comprising
a recessed portion for holding a halfway portion of the insertion portion by an elastic force, which is provided on the outer peripheral surface of the plane portion.

21. The endoscope apparatus according to claim 11, further comprising
a winding portion holding portion for bundling a spiral-shaped insertion portion having a spiral shape which is formed at a halfway portion of the insertion portion, the insertion portion being passed through one of the through holes of the insertion portion holder and the distal end side part of the insertion portion being held in the other of the through holes.

22. The endoscope apparatus according to claim 21, wherein
a function of the winding portion holding portion is performed by an attaching/detaching mechanism which enables the first housing portion and the second housing portion to be integrally attached to each other and enables the integrally attached first housing portion and the second housing portion to be separated from each other again, and
the spiral-shaped insertion portion is held in a hanging manner between the first housing portion and the second housing portion which are integrally attached to each other by the attaching/detaching mechanism.

23. The endoscope apparatus according to claim 11, wherein the winding portion holding portion is a tying member provided to the endoscope insertion portion or either one of the housing portions.

24. A method of bundling an insertion portion of an endoscope apparatus comprising steps of:
preparing an insertion portion holder including a plane portion formed in a flat shape with an elastic member, a first through hole formed on the plane portion, for passing therethrough an insertion portion having flexibility extended from an endoscope operation portion, and a second through hole formed on the plane portion adjacently to the first through hole, the second through hole including a holding portion for holding a distal end side part of the insertion portion;
passing the insertion portion through the first through hole of the insertion portion holder and moving the insertion portion holder along a longitudinal axis of the insertion portion to allow the insertion portion holder to be retained at the endoscope operation portion;
rolling up the insertion portion having flexibility into a spiral and forming a spiral-shaped insertion portion; and
arranging the distal end side part of the insertion portion in the holding portion of the second through hole in order to hold the insertion portion.

25. A method of bundling an insertion portion of an endoscope apparatus, the endoscope apparatus including: an elongated endoscope insertion portion including at a distal end side thereof an illumination portion and an observation portion; an operation portion to which a proximal end portion of the endoscope insertion portion is connected; an apparatus main body electrically connected to the operation portion; and an attaching/detaching mechanism for enabling the operation portion and the apparatus main body to be detachably attached to each other, the method of bundling the insertion portion comprising steps of:
preparing an insertion portion holder including a plane portion formed in a flat shape with an elastic member, a first through hole formed on the plane portion, for passing therethrough the insertion portion having flexibility extended from the operation portion of the endoscope, and a second through hole formed on the plane portion adjacently to the first through hole, the second through hole including a holding portion for holding a distal end side part of the insertion portion;
passing the insertion portion through the first through hole of the insertion portion holder and moving the insertion portion holder along a longitudinal axis of the insertion portion to allow the insertion portion holder to be retained at the operation portion;
attaching the operation portion and the apparatus main body integrally to each other by means of the attaching/detaching mechanism;
rolling up the insertion portion having flexibility into a spiral and forming a spiral-shaped insertion portion;
arranging the distal end side part of the insertion portion in the holding portion of the second through hole in order to hold the spiral-shaped insertion portion; and
arranging the spiral-shaped insertion portion on the attaching/detaching mechanism which attaches the operation portion and the apparatus main body integrally to each other, in order to hold the insertion portion.

26. A method of bundling an insertion portion of an endoscope apparatus, the endoscope apparatus including: an elongated endoscope insertion portion having at a distal end side thereof an illumination portion and an observation portion; an operation portion to which a proximal end portion of the endoscope insertion portion is connected; an apparatus main body electrically connected to the operation portion; and an attaching/detaching mechanism for enabling the operation portion and the apparatus main body to be detachably attached to each other, the method of bundling the insertion portion comprising steps of:
preparing an insertion portion holder including a plane portion formed in a flat shape with an elastic member, a first through hole formed on the plane portion, for passing therethrough the insertion portion having flexibility extended from the operation portion of the endoscope, and a second through hole formed on the plane portion adjacently to the first through hole, the second through hole including a holding portion for holding a distal end side part of the insertion portion;
passing the insertion portion through the first through hole of the insertion portion holder and arranging the insertion portion holder between a distal end and a proximal end of the insertion portion;
attaching the operation portion and the apparatus main body integrally to each other by means of the attaching/detaching mechanism;
rolling up the insertion portion into a spiral by repeatedly passing the insertion portion through between the operation portion and the apparatus main body and forming a spiral-shaped insertion portion, in order to hold the insertion portion having flexibility; and
arranging the distal end side part of the insertion portion in the holding portion of the second through hole by moving the insertion portion holder along a longitudinal axis direction of the insertion portion or retaining the insertion portion holder between the distal end and the proximal end of the insertion portion, in order to hold the spiral-shaped insertion portion.

27. A method of bundling an insertion portion of an endoscope apparatus, the endoscope apparatus including: an elongated endoscope insertion portion including at a distal end side thereof an illumination portion and an observation portion; an operation portion to which a proximal end portion of the endoscope insertion portion is connected; an apparatus main body electrically connected to the operation portion; and an attaching/detaching mechanism for enabling the operation portion and the apparatus main body to be detachably attached to each other, the method of bundling the insertion portion comprising steps of:
preparing an insertion portion holder including a plane portion formed in a flat shape with an elastic member, a first through hole formed on the plane portion, for passing therethrough the insertion portion having flexibility extended from the operation portion of the endoscope, and a second through hole formed on the plane portion adjacently to the first through hole, the second through hole having a first holding portion and a second holding portion, the first holding portion having a larger diameter than that of the second holding portion and the second holding portion being for holding a distal end side part of the insertion portion;
passing the insertion portion through the first through hole of the insertion portion holder and arranging the insertion portion holder between a distal end and a proximal end of the insertion portion;
attaching the operation portion and the apparatus main body integrally to each other by means of the attaching/detaching mechanism;
rolling up the insertion portion into a spiral by repeatedly passing the insertion portion through between the operation portion and the apparatus main body and by passing the insertion portion also through the first through hole, and forming a spiral-shaped insertion portion, in order to hold the insertion portion having flexibility; and
arranging the distal end side part of the insertion portion in the second holding portion of the second through hole by moving the insertion portion holder along a longitudinal axis direction of the insertion portion or retaining the insertion portion holder between the distal end and the proximal end of the insertion portion, in order to hold the spiral-shaped insertion portion.

28. A method of bundling an insertion portion of an endoscope apparatus, the endoscope apparatus including: an elongated endoscope insertion portion including at a distal end side thereof an illumination portion and an observation portion; an operation portion to which a proximal end portion of the endoscope insertion portion is connected; an apparatus main body electrically connected to the operation portion; and an attaching/detaching mechanism for enabling the operation portion and the apparatus main body to be detachably attached to each other, the method of bundling the insertion portion comprising steps of:
preparing an insertion portion holder including a plane portion formed in a flat shape with an elastic member, a first through hole which is formed on the plane portion and which can hold the insertion portion having flexibility extended from the operation portion of the endoscope, and a second through hole formed on the plane portion adjacently to the first through hole, for holding a distal end side part of the insertion portion;
passing the insertion portion through the first through hole of the insertion portion holder and arranging the insertion portion holder on the insertion portion;
attaching the operation portion and the apparatus main body integrally to each other by means of the attaching/detaching mechanism;
passing the insertion portion through between the operation portion and the apparatus main body repeatedly and forming a spiral-shaped insertion portion, in order to hold the insertion portion having flexibility; and
arranging the distal end side part of the insertion portion in the second through hole so as to be held therein by moving the insertion portion holder along a longitudinal axis direction of the insertion portion against an elastic force of the insertion portion holder or retaining the insertion portion holder on the insertion portion, in order to hold the spiral-shaped insertion portion.

29. A method of bundling an insertion portion of an endoscope apparatus, the endoscope apparatus including: an elongated endoscope insertion portion including at a distal end side thereof an illumination portion and an observation portion; an operation portion to which a proximal end portion of the endoscope insertion portion is connected; an apparatus main body electrically connected to the operation portion; an attaching/detaching mechanism for enabling the operation portion and the apparatus main body to be detachably attached to each other; the method of bundling the insertion portion comprising steps of:
preparing an insertion portion holder including a plane portion formed in a flat shape with an elastic member, a first through hole formed on the plane portion, for passing therethrough the insertion portion having flexibility extended from the operation portion of the endoscope, a second through hole formed on the plane portion adjacently to the first through hole, for holding a distal end side part of the insertion portion, and a holding groove for holding the insertion portion, the holding groove being formed on an outer peripheral surface of the plane portion;
passing the insertion portion through the first through hole of the insertion portion holder and arranging the insertion portion holder between a distal end and a proximal end of the insertion portion;
attaching the operation portion and the apparatus main body integrally to each other by means of the attaching/detaching mechanism;
passing the insertion portion through between the operation portion and the apparatus main body repeatedly, causing the holding groove to hold the insertion portion, and forming a spiral-shaped insertion portion, in order to hold the insertion portion having flexibility; and
arranging the distal end side part of the insertion portion in the second through hole by moving the insertion portion holder along a longitudinal direction of the insertion portion or retaining the insertion portion holder between the distal end and the proximal end of the insertion portion, in order to hold the spiral-shaped insertion portion.

30. A method of bundling an insertion portion of an endoscope apparatus, the endoscope apparatus including: an elongated endoscope insertion portion including at a distal end side thereof an illumination portion and an observation portion; an operation portion to which a proximal end portion of the endoscope insertion portion is connected, the operation portion including a grasping portion at which a tying member is provided; an apparatus main body electrically connected to the operation portion; an attaching/detaching mechanism for enabling the operation portion and the apparatus main body to be detachably attached to each other, the method of bundling the insertion portion comprising steps of:
preparing an insertion portion holder including a plane portion formed in a flat shape with an elastic member, a first through hole formed on the plane portion, for passing therethrough the insertion portion having flexibility extended from the operation portion of the endoscope, and a second through hole formed on the plane portion adjacently to the first through hole, the second through hole including a holding portion for holding the distal end side part of the insertion portion;
preparing a tying member configured of a strap having at one end side a through hole;
passing the insertion portion through the first through hole of the insertion portion holder and moving the insertion portion holder along a longitudinal axis of the insertion portion to allow the insertion portion holder to be retained at the operation portion;
passing the insertion portion through the through hole of the tying member and arranging the tying member between a distal end and a proximal end of the insertion portion;
rolling up the insertion portion having flexibility into a spiral and forming a spiral-shaped insertion portion;
winding the tying member arranged on the insertion portion around the spiral-shaped insertion portion, in order to hold the spiral shape of the spiral-shaped insertion portion;
winding the tying member arranged at the grasping portion around the spiral-shaped insertion portion, in order to hold the spiral-shaped insertion portion; and
arranging the distal end side part of the insertion portion in the second through hole, in order to hold the spiral-shaped insertion portion.
